(19) United States
(12) Patent Application Publication
(10) Pub. No.: US 2022/0160283 A1
(43) Pub. Date: May 26, 2022

(54) TRANSMEMBRANE SENSOR TO EVALUATE NEUROMUSCULAR FUNCTION

(71) Applicant: Powell Mansfield, Inc., San Diego, CA (US)

(72) Inventors: Perry T. Mansfield, Coronado, CA (US); Christopher D. Studer, La Jolla, CA (US); Gabriel Martinez-Tribolet, Chula Vista, CA (US); William Ronald Boyd, Escondido, CA (US)

(73) Assignee: Powell Mansfield, Inc., San Diego, CA (US)

(21) Appl. No.: 17/616,986

(22) PCT Filed: Jun. 4, 2020
(86) PCT No.: PCT/US2020/036175

§ 371 Date: Dec. 6, 2021

Related U.S. Application Data
(60) Provisional application No. 62/858,269, filed on Jun. 6, 2019.

(51) Int. Cl. A61B 5/313; A61B 5/301; A61B 5/00; A61B 5/296; A61B 5/257; A61B 5/273; A61B 5/30

(52) U.S. Cl. CPC A61B 5/313; A61B 5/301; A61B 5/6852; A61B 5/303; A61B 5/257; A61B 5/273; A61B 5/296

(57) ABSTRACT

Devices, systems, and methods herein relate to electromyography (EMG) that may be used in diagnostic and/or therapeutic applications, including but not limited to electrophysiological study of muscles in the body relating to neuromuscular function and/or disorders. Sensor assemblies and methods are described herein for non-invasively generating an EMG signal corresponding to muscle tissue where the sensor may be positioned directly on a surface of the muscle tissue including any associated membrane (e.g., mucosal, endothelial, synovial) overlying the muscle tissue. A sensor assembly may include one or more pairs of closely spaced, atraumatic electrodes in a bipolar or multipolar configuration. The first and second electrodes may be applied against a surface of muscle tissue (that may include a membrane overlying the muscle) and receive electrical activity signal data corresponding to an electrical potential difference of the portion of muscle between the electrodes.

### CROSS-REFERENCE TO RELATED APPLICATIONS

This application claims priority to U.S. application Ser. No. 62/858,269, filed Jun. 6, 2019, which is incorporated herein by reference in its entirety.

This application is related to PCT Application No. PCT/US2018/036151, filed on Jun. 5, 2018, which claims priority to U.S. application Ser. No. 62/515,364, filed on Jun. 5, 2017, which are hereby incorporated by reference in their entirety.

### FIELD

Devices, systems, and methods herein relate to electromyography (EMG) that may be used in diagnostic and/or therapeutic applications, including but not limited to electrophysiological study of muscles in the body relating to neuromuscular function and/or disorders.

### BACKGROUND

The human body contains more than 600 skeletal muscles as well as cardiac and smooth muscles with different properties and different selective vulnerability for certain disease processes, owing to structural or physiological differences. Disease processes that affect the limb muscles differ from those affecting cardiac, gastrointestinal, or urogenital muscles. From a practical and clinical point of view, it is helpful in most clinical scenarios to be able to confirm and characterize the nature of muscle disease, to provide appropriate and effective treatment if available.

EMG relates to the study of electrical activity occurring in peripheral nerve and muscle tissue. EMG) is the electrophysiological examination of muscle used for the diagnosis of suspected neuromuscular (NM) disorders such as myopathies, neuropathies, neuronopathies and neuromuscular junction disorders. There are typically two types of techniques for recording EMG signals: intramuscular or needle EMG (NEMG) and surface EMG (SEMG). A needle EMG procedure includes inserting a needle electrode directly into the muscle to be examined. Needle EMG is considered the clinical gold standard for assessing an array of neurophysiologic characteristics of muscle tissue for neuromuscular disease and may provide data related to the muscles and nerves (e.g., motor neurons) that control them. For example, NEMG data may permit characterization of neuromuscular function including spontaneous activity, motor unit action potential (MUAP) recruitment, activation, and morphology. However, NEMG is an invasive procedure that necessarily penetrates tissue and which may cause pain, as well as increase the risk of infection and disease transmission. Routine needle electromyography studies (NEMC) are mainly restricted to skeletal limb muscles, which are easily accessible, and are not performed routinely on less accessible muscles, such as striated muscles of the oropharynx. For example, needle insertion may cause swelling and bleeding, and in some instances, viscus perforation. Some areas of the body may be particularly sensitive to insertion of a needle electrode such as the mouth, pharynx, eyes, ears, gastrointestinal (GI) tract, urinary system, myocardium, and the like. This limits the evaluation of neuromuscular involvement in disorders involving muscles of the oropharynx such as obstructive sleep apnea (OSA) and neurogenic oropharyngeal dysphagia.

SEMG is a non-invasive and pain free EMG technique that may be used to assess muscle function by receiving electrical activity of one or more muscles from surface electrodes placed on the skin above the muscles to be examined. Surface EMG signals may be recorded over a prolonged period of time from many sites and motor units, and even when the patient is undergoing physical activity. Surface EMG is considered an acceptable technique for kinesiologic analysis of movement disorders. However, SEMG data may have limited spatial resolution relative to NEMG data due to the large surface area of SEMG sensors. For example, SEMG data may be susceptible to mechanical and electrical artifacts as well as cross-talk between adjacent muscles. Therefore, typical SEMG techniques do not reliably permit characterization of insertional activity, spontaneous activity, motor unit size and shape, and/or interference pattern. The American Academy of Neurology has concluded that SEMG is substantially inferior to NEMG for the evaluation of neuromuscular disorders. Furthermore, SEMG has limited spatial resolution, is more susceptible to mechanical and electrical artifact, and is more likely to show cross-talk between adjacent muscles than NEMG. Finally, SEMG uses adhesive surface electrodes that are not appropriate for muscles located within internal body cavities and organ systems with moist mucous membranes. Development of surface sensors and probes for internal use has not been successful so far.

Dysphagia is difficulty swallowing due to structural or functional impairment of the aerodigestive tract. Such impairment may arise from an underlying neurological disorder producing oropharyngeal dysphagia (OPD). Participants with OPD have difficulty initiating swallowing and present with choking, nasal regurgitation or coughing episodes while eating, and drooling due to difficulty managing saliva. In addition, neurogenic OPD may be complicated by aspiration pneumonia, dehydration and malnutrition, increasing the morbidity of the underlying neurological disorder. Participants with symptoms of oropharyngeal dysphagia undergo a VFSE, which in the case of neurogenic OPD, typically reveals impairment of oropharyngeal motor performance and/or laryngeal protection.

A videofluoroscopic swallowing evaluation (VFSE) is currently considered the diagnostic gold standard for accurate assessment of oropharyngeal dysphagia arising from pharyngeal muscle dysfunction due to multiple etiologies including neurologic disorders such as stroke, Parkinson's disease (PD), multiple sclerosis (MS), multi-system atrophy (MSA), and amyotrophic lateral sclerosis (ALS); autoimmune disorders such as myasthenia gravis; and myopathies such as muscular dystrophy. Both PSG and VFSE are laborious, costly, and have limited accessibility for the diagnosis of muscular disorders of the oropharynx.

OSA is a collective term used for conditions that over time cause damage to the delicate soft tissues of the upper airway from turbulent airflow and can comprise anatomically obstructive processes that result in nocturnal narrowing of the upper airway leading to partial or complete obstruction of the airway. The upper airway encompasses the entire upper airway passages to include the nasal cavity, oropharynx and hypo-pharynx. Partial and total airway obstruction results in sleep arousals, sleep fragmentation and subsequent behavioral derangements such as excessive daytime sleepiness. Concurrently, pathophysiologic derangements usually accompany the behavioral decrements with altered daytime performance and excessive daytime sleepiness. The cardiovascular derangements can cause in part high blood pressure, stroke, myocardial infarction and death. Decreased quality of life and a shortened life span are common in participants with untreated OSA.

Although the complications of OSA are well known, the physiological mechanism for upper airway collapse during inspiration and sleep is not clear. Nonetheless, there is substantial physiological, electrophysiological and histological evidence for neuromuscular impairment in the upper airway in participants with OSA, and pharyngeal nerve injury has been proposed as a possible contributory factor in OSA development, which might be related to vibratory trauma caused by sleep-disordered breathing. Evidence for sensory involvement in participants with OSA includes the finding of abnormal two-point palatal sensory discrimination, and disordered thresholds for warmth and cold detection in the oropharyngeal mucosa. Although the activity of the genioglossus, an upper airway dilator muscle, was found to be significantly greater during wakefulness in OSA, possibly due to a reflex-driven neuromuscular compensation for an anatomically compromised airway, a greater decline was observed during the early and late sleep onset period, suggesting the loss of this reflex. Evidence for motor nerve fiber loss was shown by needle electromyography (EMG), demonstrating longer duration motor unit potentials (MUP) with larger size index. Histological evidence for neuromuscular impairment in the upper airways includes frequent focal degeneration of myelinated nerve fibers and axons, increased number of sensory and motor nerve fibers, indicating peripheral sprouting secondary to neuropathy, abnormal muscle fiber variability with increased amount of connective tissue, and alternation in myosin heavy chain compositions.

In-laboratory polysomnography (PSG) is considered the gold standard for diagnosis of OSA. PSG measures numerous sleep metrics including oxygen level, sleep stages, REM patterns, wakefulness, EKG, and leg movements. An apneic event may be defined by a 3% drop in the level of oxygen associated with a pause in breathing of 10 seconds or longer. The diagnosis of OSA is determined by the apnea/hypopnea index (AHI) [normal≤5, mild OSA 5-15, moderate 15-30 and severe OSA>30].

Therefore, additional devices, systems, and methods for performing electromyography may be desirable.

### SUMMARY

Described herein are sensor assemblies and methods for non-invasively generating an EMG signal corresponding to muscle tissue where the sensor may be positioned directly on a surface of the muscle tissue including any associated membrane (e.g., mucosal, endothelial, synovial), dermal tissue or connective tissue overlying the muscle tissue. These systems and methods may also be used to permit evaluation of neuromuscular function and/or diagnosis of neuromuscular conditions associated with muscle tissue located within a moist body cavity. Conventional non-invasive EMG devices and techniques such as SEMG record electrical activity of a large surface area corresponding to muscle tissue and may have limited accuracy and utility due to muscle cross-talk (e.g., electrical interference from adjacent muscles) and noise due to moisture between a sensor and tissue (e.g., muscle having a mucosal lining). On the other hand, conventional invasive EMG devices and techniques such as NEMG may cause pain and/or damage to muscle tissue, thereby limiting their use in sensitive tissue systems (e.g., internal organ systems) and adding procedural complexity (e.g., use of general anesthesia).

Generally, the systems and methods described herein may use a sensor to contact an intact tissue surface to receive electrical activity signal data of a specific muscle through any overlying membrane without penetrating or piercing a surface of the tissue. The sensor may include a pair of rounded electrodes configured to directly press against and elastically deform the tissue surface so as to form a temporary indentation while the sensor receives electrical activity data of muscle underlying the surface. The sensor may be configured to provide repeatable signal measurements of an isolated muscle rather than a broader surface area encompassing a group of muscles. Neuromuscular function may be characterized and evaluated using the acquired sensor data.

In some variations, a diagnostic kit is provided. The kit includes a sensor assembly comprising a sensor including a first electrode, a second electrode, and a sensor housing coupling the first and second electrodes. The first and second electrodes may project from a surface of the sensor housing for a projection length and are spaced apart by a spacing distance. A first ratio of the spacing distance to the projection length may be between about 0.075:1 and about 1.5:1. The sensor assembly may further comprise a multi-conductor cable, with a first conductor electrically coupled distally to the first electrode and proximally to a first touch proof, single pole connector, a second conductor electrically coupled distally to the second electrode and proximally to a second touch proof, single pole connector, and a third conductor with a distal end terminating internally within the cable and a proximal end electrically to a third connector. The kit may further comprise a an ground adhesive patch electrode, comprising a removable release layer attached to an adhesive, a fourth proximal end electrically coupled to a fourth touch proof, single pole connector; and a fifth proximal end electrically coupled to a fifth connector that is configured to attach to the third connector of the sensor assembly.

In some of these variations, the first ratio may be between about 0.15:1 and about 0.75:1. In some variations, a second ratio of a diameter of the first and second electrodes to the spacing distance may be between about 0.2:1 and about 5:1. In some of these variations, the second ratio may be between about 0.4:1 and about 2.5:1. In some variations, a third ratio of a diameter of the first and second electrodes to the projection length may be between about 0.075:1 and about 1.5:1. In some of these variations, the third ratio may be between about 0.15:1 and about 0.75:1.

In some variations, the first and second electrodes may each comprise a rounded distal end. The first and second electrodes may be in parallel. The sensor housing may be configured to electrically isolate the first electrode from the second electrode. The first electrode may be configured as a reference electrode and the second electrode may be configured as an active electrode. The spacing distance may be between about 0.2 mm and about 1.0 mm. The projection length may be between about 0.5 mm and about 3 mm.

In some variations, a diagnostic kit is provided. The kit includes a sensor assembly comprising a sensor including a first electrode, a second electrode, and a sensor housing coupling the first and second electrodes. The first and second electrodes may project in parallel from a surface of the sensor housing. A distance between central longitudinal axes of the first and second electrodes may be between about 0.30 mm and about 2.0 mm. The sensor assembly may further comprise a multi-conductor cable, with a first conductor electrically coupled distally to the first electrode and proximally to a first touch proof, single pole connector, a second conductor electrically coupled distally to the second electrode and proximally to a second touch proof, single pole connector, and a third conductor with a distal end terminating internally within the cable and a proximal end electrically to a third connector. The kit may further comprise a an ground adhesive patch electrode, comprising a removable release layer attached to an adhesive, a fourth proximal end electrically coupled to a fourth touch proof, single pole connector; and a fifth proximal end electrically coupled to a fifth connector that is configured to attach to the third connector of the sensor assembly.

In some variations, the first and second electrodes may project from the surface of the housing for a projection length between about 0.5 mm and about 3 mm. A diameter of the first and second electrodes may be between about 0.1 mm and about 1.0 mm. The distance may be between about 0.60 mm and about 1.5 mm.

In some variations, the sensor assembly may comprise a probe comprising one or more of the sensors and a handle portion. The probe may comprise a first portion and a second portion detachably attached to the first portion. In some of these variations, the first portion may comprise a paddle shape and a radius of curvature of between about 10 cm and about 20 cm. Adjacent sensors may be spaced apart from each other between about 0.5 cm and about 5 cm. In some of these variations, the probe may comprise one or more dental markers. In some variations, the probe may further comprise a rigid catheter. In other variations, the probe may further comprise a flexible catheter.

In some variations, the assembly may further comprise an amplifier coupled to the probe. The amplifier may comprise a pre-amplifier and/or a main amplifier. A controller may be coupled to the probe and the amplifier. The controller may comprise a processor and a memory. The controller may be configured to receive signal data corresponding to electrical activity of muscle tissue using the one or more sensors. The signal data may be amplified and used to generate electromyography data.

Also described here are methods for using a sensor probe. In general, these methods include the steps of advancing and positioning the probe into a body cavity and sensing activity in the muscle tissue using the sensor. The probe may comprise one or more sensors each comprising a first electrode, a second electrode, and a sensor housing coupling the first and second electrodes. The first and second electrodes may project from a surface of the sensor housing for a projection length and may be spaced apart by a spacing distance. A first ratio of the spacing distance to the projection length may be between about 0.075:1 and about 1.5:1. One or more sensors of the probe may be applied directly on an intact tissue surface so as to elastically deform the tissue surface. Signal data corresponding to electrical activity of tissue may be received using one or more sensors without penetrating or piercing the intact tissue surface.

In some variations, the tissue surface may comprise a membrane overlying the tissue surface. The tissue surface may be maintained in an unbroken state while applying one or more sensors of the probe directly on the tissue surface. The signal data may be processed and used to generate electromyography data.

### BRIEF DESCRIPTION OF DRAWINGS

FIGS. 1A-1B are illustrative views of an exemplary variation of a bipolar sensor. FIG. 1A is a perspective view and FIG. 1B is a cross-sectional side view.

FIG. 2A is a side view of another embodiment of a rigid EMG probe. FIG. 2B is a cross-sectional view the probe in FIG. 2A and FIG. 2C is a detailed cross sectional view of a distal portion of the probe. FIG. 2D is an exploded component view of the probe in FIG. 2A.

FIGS. 3A-3C are illustrative views of another variation of a sensor assembly. FIG. 3A is a perspective view, FIG. 3B is a side view, and FIG. 3C is a detailed cross-sectional side view of the bipolar sensor depicted in FIG. 3A.

FIGS. 4A-4B are illustrative perspective views of another variation of a sensor assembly and an endoscope. FIG. 4B is a detailed perspective view of the bipolar sensor depicted in FIG. 4A.

FIG. 5 is a block diagram of another variation of a sensor assembly.

FIG. 6 is an illustrative flowchart of a variation of a method of using a sensor probe.

FIG. 7 is an illustrative frontal surface view of the oropharynx.

FIGS. 8A-8B are illustrative views of a hypopharynx. FIG. 8A is an axial surface view of the hypopharynx and FIG. 8B is an axial view of the hypopharynx musculature.

FIG. 9 is an illustrative sagittal cross-sectional view of the hypopharynx and larynx.

FIG. 10 is an illustrative cross-sectional view of the nasopharynx.

FIG. 11 is an illustrative cross-sectional view of a portion of the upper gastrointestinal tract.

FIG. 12 is an illustrative cross-sectional view of the stomach and duodenum

FIG. 13A is a graph of EMG data of a right palatoglossus muscle using an exemplary variation of a sensor assembly. FIG. 13B is a graph of EMG data of a right palatoglossus muscle using a needle electrode. FIG. 13C is a graph of EMG data of a right first dorsal interosseous muscle using a surface electrode.

FIG. 14A is a graph of motor unit action potential data of a right palatoglossus muscle using an exemplary variation of a sensor assembly. FIG. 14B is a graph of motor unit action potential data of a right palatoglossus muscle using a needle electrode. FIG. 14C is a graph of motor unit action potential data of a right first dorsal interosseous muscle using a surface electrode.

FIGS. 15A to 15D are a rear perspective, front perspective, rear end and front end views, respectively, of an exemplary probe tip cover of the probe in FIG. 2A. FIGS. 15E to 15H are top, top cross-sectional, side and side cross-sectional views, respectively, of the exemplary probe tip cover.

FIGS. 16A to 16C are perspective, side and end views, respectively, of an exemplary center section of the probe in FIGS. 2A to 2D.

FIG. 17A is a front perspective view of the back cap of the exemplary probe in FIGS. 3A to 3D. FIG. 17B and 17C are top and top cross-sectional views, respectively, of the exemplary back cap in FIG. 17A. FIGS. 17D and 17E are front and rear end views, respectively, of the exemplary back cap.

FIG. 18 is an exploded component view of an exemplary ground surface electrode that may be used with the exemplary probe.

### DETAILED DESCRIPTION

Described herein are sensor devices, systems, and methods for use in non-invasive diagnostic procedures of neuromuscular function of tissue in a body cavity or on a surface of an anatomical structure. In some variations, a sensor assembly may be used for measuring electrical activity of one or more muscles. Generally, a non-invasive transmembrane EMG (TM-EMG) sensor may be used to receive electrical activity signal data corresponding to a specific muscle, with the signal data used to generate EMG data. One or more of the sensors may be incorporated into one or more sensor arrays in a probe. The probe and sensor arrays may be configured to contact muscle tissue in a membranous body cavity (e.g., oropharynx, abdominal cavity, pelvic cavity, joint cavity) or other anatomical structure (e.g., eyes), including anatomical structures accessed intraoperatively.

A sensor assembly as described herein may include one or more pairs of closely spaced, atraumatic electrodes in a bipolar or multipolar configuration. For example, a first electrode may be configured as a reference electrode and a second electrode may be configured as an active electrode. The first and second electrodes may be applied against a surface of muscle tissue (that may include a membrane overlying the muscle) and receive electrical activity signal data corresponding to an electrical potential difference (e.g., voltage) of the portion of muscle between the electrodes. Each electrode may comprise a shape to project or extend into the target muscle tissue. For example, the electrodes may comprise a generally cylindrical shape having a semispherical distal end. The electrodes may be applied against the muscle such that muscle tissue contacts the distal end and/or distal portions of the electrode. However, the shape, length, and spacing of the electrodes are such that the contact is atraumatic and does not damage the muscle (e.g,, tear, penetrate the surface). Common noise between the first and second electrodes may be reduced due to the close spacing between the first and second electrode, thereby increasing the SNR of the signal and increasing specificity of the signal data. The atraumatic configuration of the sensor further permits stable and reproducible measurements using the sensor assemblies. Furthermore, the sensor assemblies as described herein may be used in areas of the body that are not typically assessed with NEMC and SEMG. For example, the sensor assemblies as described herein may be used within body cavities and their associated internal organ systems during a surgery or invasive procedure. For example, the sensor assemblies may contact moist muscle tissue having an overlying membrane (e.g., mucosal, endothelial, synovial).

In variations where a controller including a processor and memory are coupled to a TM-EMG sensor, the processor may generate EMG data using the signal data received from the TM-EMG sensor, EMG data generated from the sensor data may correspond to native or spontaneous neuromotor activity and/or a superposition of the evoked action potentials of the active motor units in the measured muscle. The EMG data may have a signal-to-noise ratio (SNR) that permits evaluation of neuromuscular function according to parameters such as insertional activity, spontaneous activity, motor unit size and shape, and interference pattern using sensor data acquired from the devices and systems as described herein.

In some variations, a probe having one or more sensors may be disposed in a housing (e.g., probe) having a size and shape matching a contour of the tissue to be evaluated. Intermediate and proximal portions of the probe may comprise a configuration to aid advancement of the probe to a target muscle. For example, portions of the probe may be flexible or rigid. In some of these variations, a probe may be advanced into a body cavity of interest using a delivery device such as a catheter or endoscope.

### I. Sensor

A. Electrodes

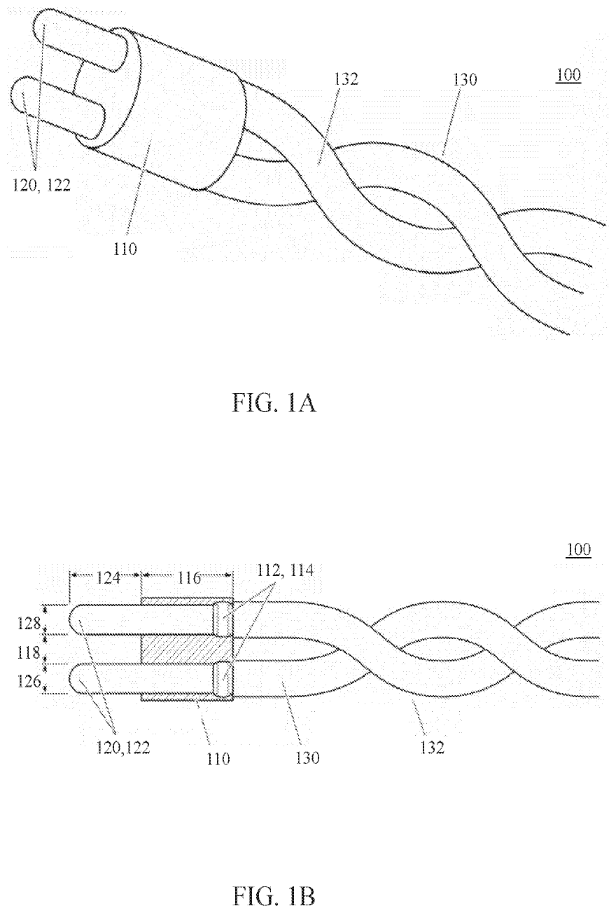

Described herein are electrode sensors for use in measuring electrical activity of one or more muscles. The electrodes may be unipolar, bipolar, or multipolar, and each electrode may comprise a different configuration. FIGS. 1A-1B are illustrative perspective and cross-sectional side views, respectively, of a bipolar sensor (100). The bipolar sensor (100) may comprise a housing (110), a first electrode (120), a second electrode (122), a first lead wire (130), and a second lead wire (132). The housing (110) may have a housing length (116). The housing (110) may couple to the first electrode (120), the second electrode (122), the first lead wire (130), and the second lead wire (132). The first electrode (120) and the second electrode (122) may each project from a surface of the housing (110) for a projection length (124) such that distal portions of the first and second electrodes (120, 122) are uncovered and exposed. The first electrode (120) may have a first diameter (126) and the second electrode (122) may have a second diameter (128). The first and second electrodes (120, 122) may be spaced apart by a spacing distance (118). A first connector (112) may couple the first electrode (120) and the first lead wire (130). A second connector (114) may couple the second electrode (122) and the second lead wire (132). For example, the first and second connectors (112, 114) may be weld points for a solder connection, a pin connector, and the like.

The first electrode (120) and the second electrode (122) may comprise an atraumatic configuration to reduce or prevent damage to tissue damage during contact and/or signal acquisition with the sensor (100). For example, each of the electrodes (120, 122) may comprise a cylindrical body and a semi-spherical or other rounded distal end. In other variations, the electrodes may comprise other shapes (e.g., rectangular body, blunted distal end, rounded edges, flat surfaces, protruding surfaces, smooth surfaces, rough surfaces, grooved surfaces, indented surfaces, mixed surfaces) that are atraumatic to tissue. As another example, one or more of the electrodes may comprise a curved shape (e.g., C-shaped) and/or one or more bends.

In some variations, the first electrode (120) and the second electrode (122) may be parallel to each other. In other variations, the first and second electrodes (120, 122) may be angled non-parallel to each other. For example, the first and second electrodes (120, 122) may form a V-shaped projection relative to each other projecting from the housing (110).

In some variations, as shown in FIGS. 1A-1B, the first and second electrodes (120, 122) may have the same configuration (e.g., dimensions, shape, and orientation). In other variations, the first and second electrodes (120, 122) may have different configurations. For example, the sensor (100) may be configured to have a shape corresponding to a muscle to be measured such that one electrode may be longer than the other electrode, and have different diameters and/or shapes. A spacing distance (118) between the electrodes (120, 122) may be based on the submucosal, subendothelial, subsynovial, muscular anatomy.

The electrodes (120, 122) of the sensor (100) may comprise dimensions such that the electrode pair is atraumatic when in contact with muscle tissue. The dimensions described herein permit the electrodes to measure electrical activity of muscle tissue. In some variations, the electrodes (120, 122) may comprise a diameter (126, 128) between about 0.1 mm and about 1.0 mm. In some variations, the electrodes (120, 122) may comprise a diameter (126, 128) between about 0.3 mm and about 0.75 mm. In some variations, the electrodes (120, 122) may comprise a projection length (124) between about 0.5 mm and about 3.0 mm. In some variations, the electrodes (120, 122) may comprise a projection length (124) between about 0.5 mm and about 2.5 mm. In some variations, the electrodes (120, 122) may comprise a projection length (124) between about 1.0 mm and about 2.0 mm. In some variations, the electrodes (120, 122) may comprise a total length (e.g., projection length and insulated length) of between about 0.5 mm and about 5.0 mm.

The dimensions described herein permit the electrodes to measure electrical activity of muscle tissue atraumatically and with specificity to evaluate neuromuscular function of desired tissue. The electrodes (120, 122) of the sensor (100) may comprise a spacing distance (118) configured such that desired muscle tissue may be isolated while permitting a potential difference of muscle between the electrodes to be measured. For example, the electrode spacing (118) of the bipolar sensor (100) disclosed herein is such that common noise between the electrodes (120, 122) may be reduced to thereby improve an SNR of the bipolar sensor signal data. For example, a smaller spacing distance (118) corresponds to a more focused and precise measurement of muscle while a larger spacing distance (118) corresponds to a more general measurement of the muscle. In some variations, the electrodes (120, 122) may comprise a spacing distance (118) between about 0.2 mm and about 1.0 mm. In some variations, the electrodes (120, 122) may comprise a spacing distance (118) between about 0.3 mm and about 0.75 mm. In other variations, the electrodes (120, 122) may comprise a spacing distance between a first central longitudinal axis (e.g., through the center or midpoint) of the first electrode and a second central longitudinal axis of the second electrode may be between about 0.3 mm and about 2.0 mm. In some other variations, the electrodes (120, 122) may comprise a spacing distance between a first central longitudinal axis of the first electrode and a second central longitudinal axis of the second electrode of between about 0.6 mm and about 1.5 mm.

The sensors described herein may permit the electrodes to measure electrical activity of muscle tissue atraumatically and with specificity to evaluate neuromuscular function based on one or more relationship(s) between the dimensions of the electrodes. For example, electrode dimensions including spacing distance, electrode length, and electrode diameter may be related such that the electrodes are spaced close enough to permit voltage measurement of desired muscle tissue and the shape and dimensions of the electrodes are atraumatic to reduce damage to tissue (e.g., tissue piercing). In some variations, a first ratio of the spacing distance (118) to the projection length (124) may be between about 0.075 and about 1.5:1. In some variations, a first ratio of the spacing distance (118) to the projection length (124) may be between about 0.15:1 and about 0.75:1. In some variations, a second ratio of a diameter of the first and second electrodes (120, 122) to the spacing distance (118) may be between about 0.2:1 and about 5:1. In some variations, the second ratio of a diameter of the first and second electrodes (120, 122) to the spacing distance (118) may be between about 0.4:1 and about 2.5:1. In some variations, a third ratio of a diameter of the first and second electrodes (120, 122) to the projection length (124) may be between about 0.075:1 and about 1.5:1. In some variations, the third ratio of a diameter of the first and second electrodes (120, 122) to the projection length (124) may be between about 0.15:1 and about 0.75:1.

The electrodes as described herein may be formed of any biocompatible conductive metal and/or alloy including, but not limited to tungsten, silver, platinum, platinum-iridium, nickel titanium alloys, copper-zinc-aluminum-nickel alloys, copper-aluminum-nickel alloys, combinations thereof, and the like. The lead wires described herein may comprise an electrically conductive wire configured to connect the electrodes of a bipolar sensor to other components of a sensor assembly, such as an amplifier, controller, and the like. The amplifier may comprise a pre-amplifier, either alone or in combination with another amplifier. In some variations, each electrode may be coupled to a respective insulated lead wire. The lead wires (130, 132) of a pair of electrodes (120, 122) may be configured as a twisted pair (e.g., braided). This twisting may reduce the electromagnetic interference and/or crosstalk from other pairs of lead wires in the device. The number of twists per inch may be in the range of about 0.5 to about 5 twists per inch, and different pairs may have different twists per inch. The lead wires as described herein may comprise any length necessary to couple its corresponding electrode to the sensor assembly. In some variations, the lead wire may comprise a length of between about 0.1 m and about 2.0 m. In some variations, the lead wire may comprise a length of between about 0.5 m and about 1.5 m. In some variations, the lead wire may have about the same diameter as its corresponding electrode. The lead wires as described herein may be formed of any electrically conductive metal and/or biocompatible conductive metal and/or alloy including, but not limited to copper, silver, platinum, platinum-iridium, combinations thereof, and the like. In some variations, the lead wires may comprise a touch proof, single pole connector (e.g., DIN 42-802) at a proximal end. In some variations, the lead wires may be stranded or solid.

One or more portions of the lead wires may be flexible or semi-flexible, one or more portions may be rigid or semi-rigid, and/or one or more portions of the lead wires may transition between flexible and rigid configurations. The lead wires described herein may be made of any material or combination of materials. For example, the lead wires may be insulated using one or more polymers (e.g., silicone, polyvinyl chloride, latex, polyurethane, polyethylene, PTFE, nylon).

In some variations, the sensors described herein may comprise a ground electrode and a corresponding ground wire configured to reduce noise. The ground electrode and ground wire may be separate from or integrated with the sensor in a housing. The ground electrode and ground wire may be formed of any biocompatible conductive metal and/or alloy including, but not limited to tungsten, silver, platinum, platinum-iridium, nickel titanium alloys, copper-zinc-aluminum-nickel alloys, copper-aluminum-nickel alloys, combinations thereof, and the like.

In some variations, the sensor may be configured as a multipolar sensor with three or more of the electrodes as described herein. The electrodes of a multipolar sensor may be configured to optimize surface area contact with predetermined muscle tissue, thereby increasing the SNR of the signal and specificity of the signal data.

In some variations, the sensor electrodes, ground electrode and lead wires as described herein may be integrated into a single cable. For example, the cable may comprise one or more layers of shielding and insulation. In some variations, the shielding and insulation layers may be disposed individually over one or more of the sensor and ground electrodes and/or disposed over the cable as a whole. The ground electrode of a single cable may comprise an interwoven mesh or spiral shape with helical, wrapped strands. In some variations, the cable may comprise one or more ground electrodes. For example, the cable may comprise a ground electrode for each sensor electrode. The lead wires of the cable may be stranded or solid. For example, the number of strands may be between about 7 and about 100.

B. Housing

As shown in FIGS. 1A-1B, the bipolar sensor (100) may comprise a housing (110) configured to physically support and/or protect the electrodes (120, 122), lead wires (130, 132), and connectors (112, 114) coupled therebetween. The housing (110) may be further configured to electrically isolate the first electrode (120) from the second electrode (122). The housing (110) may have any atraumatic configuration that does not damage muscle tissue. The housing (110) may be configured to have any length to support and/or protect the electrodes (120, 122), connectors (112, 114), and lead wires (130, 132), and may be based on the muscle to be evaluated. In some variations, the housing (110) may comprise a length of between about 1.0 mm and about 2.0 mm. In some variations, the housing (110) may comprise a diameter to surround the pair of spaced-apart electrodes (120, 122). The housing as described herein may be formed of any biocompatible non-conductive material including, but not limited to epoxy, Teflon, PVS, ABS plastic, silicone, polyvinyl chloride, latex, polyurethane, polyethylene, PTFE, nylon, combinations thereof, and the like.

### II. Sensor Assembly

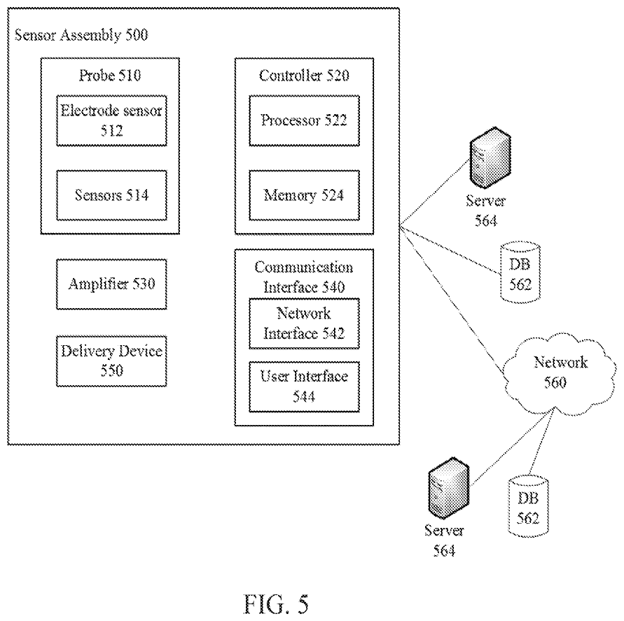

A sensor assembly may include one or more of the components necessary to measure and evaluate muscle tissue using the bipolar or multipolar sensors as described herein. The sensor assembly may couple to one or more computer systems and/or networks. FIG. 5 is a block diagram of another variation of a sensor assembly (500). The sensor assembly (500) may comprise a probe (510) that may be advanced into a body cavity or surface of an anatomical structure and placed against muscle to be evaluated. In some variations, the probe (510) may comprise one or more electrode sensors (512) and/or additional sensors (514). In some variations, the additional sensors (514) may comprise one or more of a thermal sensor, optical sensor (e.g., CCD), light source, proximity sensor, and the like. For example, an optical sensor may permit visualization of a body cavity or anatomical surface that may aid probe placement. The probe (510) may be coupled to a controller (520) configured to receive and process the sensor data from the probe (510). The controller (520) may comprise a processor (522) and a memory (524). In some variations, the sensor assembly (500) may further comprise one or more of an amplifier (530), a communication interface (540), and a delivery device (550). The probe (510) and controller (520) may be coupled to the amplifier (530) that is configured to process the electrode sensor signal data to, for example, increase the SNR of the signal data. The controller (520) may be coupled to the communication interface (540) to permit an operator to control the sensor assembly (500), probe (510), signal processing, data output, etc. The communication interface (540) may comprise a network interface (542) configured to connect the sensor assembly (500) to another system (e.g., Internet, remote server, database) over a wired and/or wireless network. The communication interface (540) may further comprise a user interface (544) configured to permit an operator to directly control the sensor assembly (500). In some variations, the probe (510) may be advanced into a body cavity using a delivery device (550) such as a catheter or endoscope.

A. Controller

A sensor assembly (500), as depicted in FIG. 5, may comprise a controller (520) in communication with one or more probes (510). The controller (520) may comprise one or more processors (522) and one or more machine-readable memories (524) in communication with the one or more processors (522). The processor (522) may incorporate data received from memory (524) and operator input to control the sensor assembly (500) (e.g., one or more probes (510) and/or delivery devices (550)). The memory (524) may further store instructions to cause the processor (522) to execute modules, processes, and/or functions associated with the sensor assembly (500). The controller (520) may be connected to the one or more probes (510) by wired or wireless communication channels. In some variations, the controller (520) may be coupled to a patient platform or disposed on a medical cart adjacent to the patient and/or operator. The controller (520) may be configured to control one or more components of the sensor assembly (500), such as probe (510), communication interface (540), delivery device (550), and the like.

The controller (520) may be implemented consistent with numerous general purpose or special purpose computing systems or configurations. Various exemplary computing systems, environments, and/or configurations that may be suitable for use with the systems and devices disclosed herein may include, but are not limited to software or other components within or embodied on personal computing devices, network appliances, servers or server computing devices such as routing/connectivity components, portable (e.g., hand-held) or laptop devices, multiprocessor systems, microprocessor-based systems, and distributed computing networks. Examples of portable computing devices include smartphones, personal digital assistants (PDAs), cell phones, tablet PCs, phablets (personal computing devices that are larger than a smartphone, but smaller than a tablet), wearable computers taking the form of smartwatches, portable music devices, and the like, and portable or wearable augmented reality devices that interface with an operator's environment through sensors and may use head-mounted displays for visualization, eye gaze tracking, and user input.

i. Processor

The processor (522) may be any suitable processing device configured to run and/or execute a set of instructions or code and may include one or more data processors, image processors, graphics processing units, physics processing units, digital signal processors, and/or central processing units. The processor (522) may be, for example, a general purpose processor, Field Programmable Gate Array (FPGA), an Application Specific Integrated Circuit (ARC), and/or the like. The processor (522) may be configured to run and/or execute application processes and/or other modules, processes and/or functions associated with the system and/or a network associated therewith. The underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

ii. Memory

In some variations, the memory (524) may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, and the like. As used herein, database refers to a data storage resource. The memory (524) may store instructions to cause the processor (522) to execute modules, processes and/or functions associated with the sensor assembly (500), such as probe control, signal data processing, EMG data processing, sensor control, communication, and/or user settings. In some variations, storage may be network-based and accessible for one or more authorized users, Network-based storage may be referred to as remote data storage or cloud data storage. EMG signal data stored in cloud data storage (e.g., database) may be accessible to respective users via a network, such as the Internet. In some variations, database (120) may be a cloud-based FPGA.

Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic, wave carrying information on a transmission medium such as space or a cable). The media and computer code (also may be referred to as code or algorithm) may be those designed and constructed for the specific purpose or purposes. Examples of non-transitory computer-readable media include, but are not limited to, magnetic storage media such as: hard disks; floppy disks; magnetic tape; optical storage media such as Compact Disc/Digital Video Discs (CD/DVDs); Compact Disc-Read Only Memories (CD-ROMs); holographic devices; magneto-optical storage media such as optical disks; solid state storage devices such as a solid state drive (SSD) and a solid state hybrid drive (SSW)); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

B. Amplifier

A sensor assembly (500), as depicted in FIG. 5, may comprise an amplifier (530) coupled to one or more of the probe (510), controller (520), and communication interface (540). The amplifier (530) may be configured to process electrical activity signal data. from one or more of the bipolar or multipolar sensors (512) and/or sensors (514). For example, the amplifier (530) may be configured to process the bipolar sensor signal data to improve signal-to-noise (SNR) by reducing artifacts, cross-talk, and increasing spatial resolution. In some variations, the amplifier (530) may comprise one or more of a pre-amplifier, main amplifier and a multi-stage differential amplifier. For example, a differential amplifier (530) may be configured to amplify a voltage difference measured between a pair of electrodes of a multipolar electrode sensor (512). The amplifier (530) may comprise several stages to increase the gain of the SNR ratio by amplification of the voltage signal near the source, prior to the emergence of noise that develops in circuits of the sensor assembly (500). For example, a differential amplifier may reduce artifacts due to AC power and action potentials of distant muscles. In some variations, electrode sensitivity may be set at about 50 uV/division, but in other embodiments may be between about 40 uV/division and about 60 uV/division, or between about 30 uV/division and about 100 uV/division, or between about 10 uV/division and about 200 uV/division. The sweep speeds may be set at about 10 ms/division, or may be between about 5 μV/division and about 20 ms/division, or between about 3 uV/division and about 30 ms/division. In some variations, threshold capture may be established at about 100 uV, but in other examples may be between about 50 uV and about 150 uV, or between about 80 uV and about 200 uV.

C. Communication Interface

The communication interface (544) may permit an operator to interact with and/or control the sensor assembly (500) directly and/or remotely. For example, a user interface (544) of the sensor assembly (500) may include an input device for an operator to input commands and an output device for an operator and/or other observers to receive output (e.g., view patient data on a display device) related to operation of the sensor assembly (500), in some variations, a network interface (542) may permit the sensor assembly (500) to communicate with one or more of a network (560) (e.g., Internet), remote server (564), and database (562) as described in more detail herein.

i. User Interface

User interface (544) may serve as a communication interface between an operator and the sensor assembly (500). In some variations, the user interface (544) may comprise an input device and output device (e.g., touch screen and display) and be configured to receive input data and output data from one or more of the probe (510), delivery device (550), input device, output device, network (560), database (562), and server (564). For example, images generated by an optical sensor of a delivery device (550) (e.g., an endoscope) may be processed by processor (522) and memory (524), and displayed by the output device (e.g., monitor display). Sensor data from one or more sensors (512, 514) may be received by user interface (544) and output visually and/or audibly through one or more output devices. As another example, operator control of an input device (e.g., joystick, keyboard, touch screen) may be received by user interface (544) and then processed by processor (522) and memory (524) for user interface (544) to output a control signal to one or more probes (510) and delivery devices (550).

1. Output Device

An output device of a user interface (544) may output sensor data corresponding to a patient and/or sensor assembly (500), and may comprise one or more of a display device and audio device. The output device may be coupled to a patient platform and/or disposed on a medical cart adjacent to the patient and/or operator. In other variations, the output device may be mounted to any suitable object, such as furniture (e.g., a bed rail), a wall, a ceiling, and may be self-standing.

The display device may be configured to display a graphical user interface (GUI). A display device may permit an operator to view signal data, EMG data, and/or other data processed by the controller (520) such as images of one or more body cavities and tissue. For example, an endoscope comprising an optical sensor (e.g., camera) located in a body cavity or lumen of a patient may be configured to image an internal view of the body cavity and/or muscle tissue to be measured. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

An audio device may audibly output patient data, sensor data, system data, alarms, and/or warnings. For example, the audio device may output an audible warning when monitored patient data (e.g., temperature, heart rate) falls outside a predetermined range or when a malfunction in the probe (510) is detected. In some variations, an audio device may comprise at least one of a speaker, piezoelectric audio device, magnetostrictive speaker, and/or digital speaker. In some variations, an operator may communicate with other users using the audio device and a communication channel. For example, the operator may form an audio communication channel (e.g., VoIP call) with a remote operator and/or observer.

2. Input Device

Some variations of an input device may comprise at least one switch configured to generate a control signal. The input device may be coupled to a patient platform and/or disposed on a medical cart adjacent to the patient and/or operator. However, the input device may be mounted to any suitable object, such as furniture (e.g., a bed rail), a wall, a ceiling, or may be self-standing. In some variations, the input device may comprise a wired and/or wireless transmitter configured to transmit a control signal to a wired and/or wireless receiver of a controller (520). For example, an input device may comprise a touch surface for an operator to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, pointing device (e.g., mouse), trackball, jog dial, step switch, rocker switch, pointer device (e.g, stylus), motion sensor, image sensor, and microphone. A motion sensor may receive operator movement data from an optical sensor and classify an operator gesture as a control signal. A microphone may receive audio and recognize an operator voice as a control signal.

ii. Network Interface

As depicted in FIG. 5, a sensor assembly (500) described herein may communicate with one or more networks (560) and computer systems (564) through a network interface (542), In some variations, the sensor assembly (500) may be in communication with other devices via one or more wired and/or wireless networks. The network interface (110) may facilitate communication with other devices over one or more external ports (e.g., Universal Serial Bus (USB), multi-pin connector) configured to couple directly to other devices or indirectly over a network (e.g., the Internet, wireless LAN).

In some variations, the network interface (542) may comprise a radiofrequency receiver, transmitter, and/or optical (e.g., infrared) receiver and transmitter configured to communicate with one or more devices and/or networks. The network interface (542) may communicate by wires and/or wirelessly with one or more of the probe (510), delivery device (550), user interface (544), network (560), database (562), and server (564).

In some variations, the network interface (542) may comprise radiofrequency (RF) circuitry (e.g., RF transceiver) including one or more of a receiver, transmitter, and/or optical (e.g., infrared) receiver and transmitter configured to communicate with one or more devices and/or networks. RF circuitry may receive and transmit RF signals (e.g., electromagnetic signals). The RF circuitry converts electrical signals to/from electromagnetic signals and communicates with communications networks and other communications devices via the electromagnetic signals. The RF circuitry may include one or more of an antenna system, an RF transceiver, one or more amplifiers, a tuner, one or more oscillators, a digital signal processor, a CODEC chipset, a subscriber identity module (SIM) card, memory, and the like. A wireless network may refer to any type of digital network that is not connected by cables of any kind. Examples of wireless communication in a wireless network include, but are not limited to cellular, radio, satellite, and microwave communication. The wireless communication may use any of a plurality of communications standards, protocols and technologies, including but not limited to Global System for Mobile Communications (GSM), Enhanced Data GSM Environment (EDGE), high-speed downlink packet access (HSDPA), wideband code division multiple access (W-CDMA), code division multiple access (CDMA), time division multiple access (TDMA), Bluetooth, near-field communication (NFC), radio-frequency identification (RFID), Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email (e,g., Internet Message Access Protocol (IMAP) and/or Post Office Protocol (POP)), instant messaging (e.g., eXtensible Messaging and Presence Protocol (XMPP), Session Initiation Protocol for Instant Messaging and Presence Leveraging Extensions (SIMPLE), and/or Instant Messaging and Presence Service (IMPS)), and/or Short Message Service (SMS), or any other suitable communication protocol. Some wireless network deployments combine networks from multiple cellular networks or use a mix of cellular, Wi-Fi, and satellite communication. In some variations, a wireless network may connect to a wired network in order to interface with the Internet, other carrier voice and data networks, business networks, and personal networks. A wired network is typically carried over copper twisted pair, coaxial cable, and/or fiber optic cables. There are many different types of wired networks including, but not limited to, wide area networks (WAN), metropolitan area networks (MAN), local area networks (LAN), Internet area networks (IAN), campus area networks (CAN), global area networks (GAN), like the Internet, and virtual private networks (VPN). As used herein, network refers to any combination of wireless, wired, public, and private data networks that are typically interconnected through the Internet, to provide a unified networking and information access system.

In some variations, the sensor assembly (500) may comprise an analog-to-digital converter (not shown) configured to convert an analog voltage signal into a digital voltage signal. The accuracy of the signal conversion may depend on the sampling frequency and the number of steps (e.g., vertical resolution). For example, a high sampling frequency and a large number of steps may produce a more accurate digital replica of the received analog signal.

D. Probe

A probe (510) may comprise one or more of the bipolar or multipolar electrode sensors (512) configured to measure electrical activity of a set of muscles. The probe (510) may comprise a housing configured with a size, shape, and sensor arrangement suited for advancement into a body cavity or surface of an anatomical structure and the muscle(s) to be evaluated. For example, a transoral probe comprising a curved, rigid probe housing including at least two sensor arrays may be configured to contact and measure electrical activity of a group of muscles in an upper airway cavity. In another example, a rigid probe may comprise a single bipolar electrode sensor coupled to a rigid shaft. The probe configurations as described herein are merely illustrative.

In some variations, the devices, systems, and methods may comprise one or more elements described in International Application Serial No. PCT/US2015/018196, filed on Feb. 27, 2015, and titled “SYSTEMS, METHODS AND DEVICES FOR SENSING EMG ACTIVITY,” and/or U.S. Provisional Application Ser. No. 61/946,259, filed on Feb. 28, 2014, and titled “SYSTEMS, METHODS AND DEVICES FOR SLEEP APNEA,” each of which is hereby incorporated by reference in its entirety.

i. RIGID EMG Probe

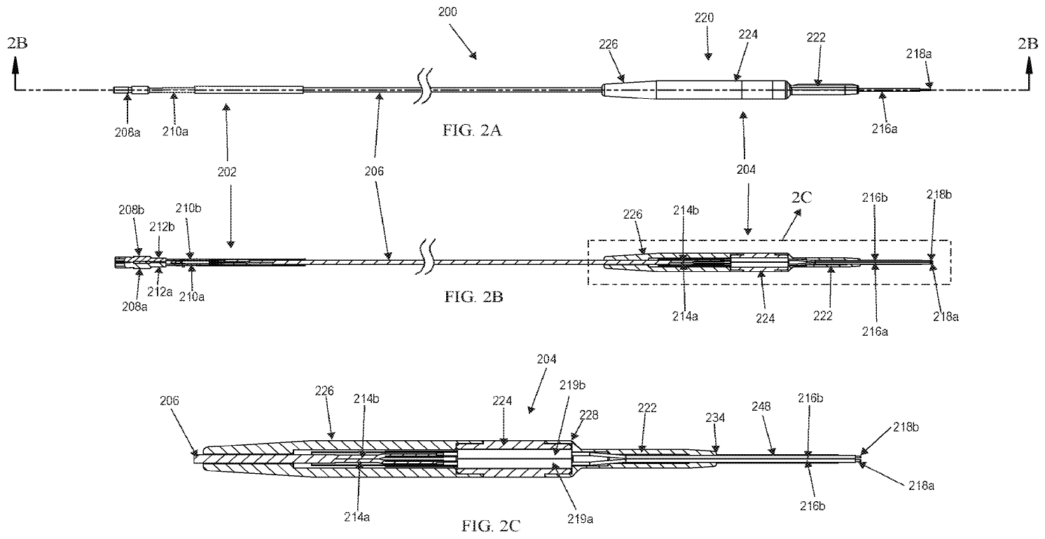

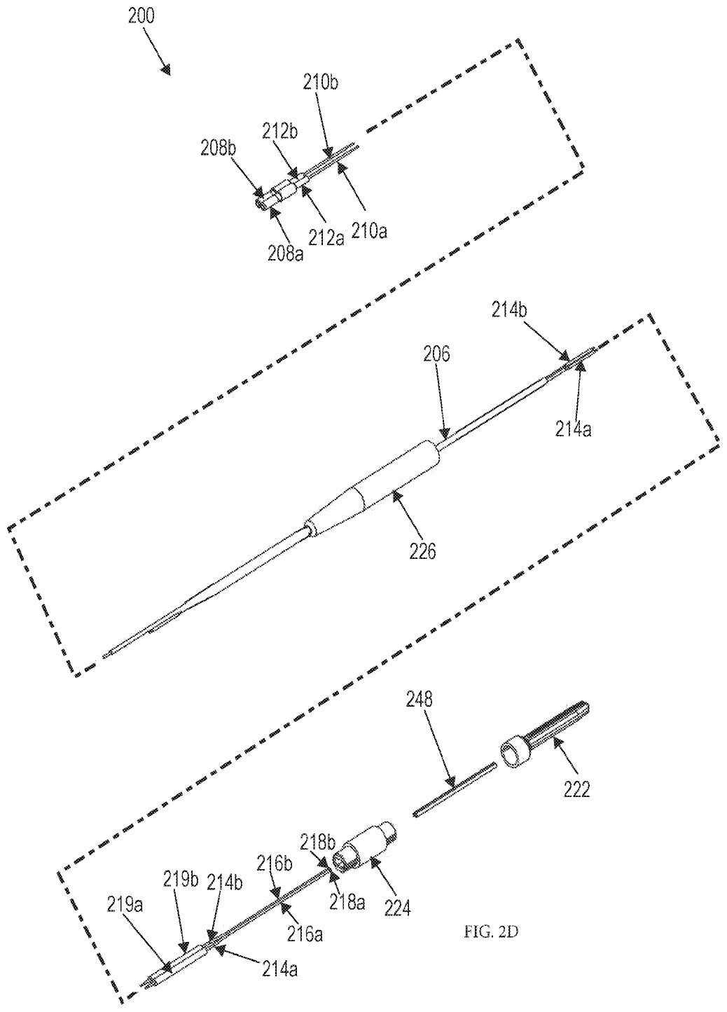

FIGS. 2A-2D are illustrative views of one example of a bipolar transoral EMG probe (200). The probe (200) may be configured for placement adjacent to at least one of the soft palate, pharyngeal wall, and tongue of a patient, as described in more detail herein. For example, the size, shape, and other physical characteristics of a probe housing may be configured for an upper airway cavity of a patient to permit evaluation of muscle tissue. The probe (200) as depicted in the side elevational view of FIG. 2A may comprise a proximal section (202) coupled to a distal section (204) via a cable (206). The proximal section (202) comprises one or more plugs (208a, 208b) that are configured to attach the probe (200) to a receiver or signal amplifier. The cable (206) may be split at its proximal end into two cable ends (210a, 210b) that are coupled to each plug (208a, 208b). The plug(s) may comprise a proprietary design or may comprise a standardized design. For example, each plugs (208a, 208b) may comprise a DIN 42-802 single pole touch proof safety connector that is commonly used as an electrical connector in EEG and EMG recording systems. The plug may comprise a distal opening configured to be inserted into an opening with a recessed metal pin, commonly a 1.5 mm pin. The distal opening communicates with a metal socket to receive the pin when inserted into the receiver or signal amplifier. The outer surface of the distal end of the plug may be configured to form a mechanical interfit or friction fit with the opening, or the metal socket may be a flexible socket that is configured to form a friction fit with the pin. The proximal end of each plug (208a, 208b) may comprise a ribbed or helical strain relief structure (212a, 212b) where the proximal cable ends (210a, 210b) attach. The cable (206) may be a jacketed, shielded, insulated, multi-conductor cable and have a length in the range of 100 cm to 400 cm, or 120 cm to 200 cm, or 150 cm to 250 cm. In other examples, the cable may comprise multiple separate conductors, or multiple individual conductors are subsequently covered with heat shrink tubing. In one particular example, the cable (206) comprises PVC outer jacket with an aluminum/polyester outer shielding, polyolefin insulation, and two conductors, each comprising multi-stranded 24 AWG tinned copper wire, e.g. Belden 1508A cable. The distal end of the cable (206) may also be split into two distal cable ends (214a, 214b) which are each attached to elongate electrode shafts (216a, 216b). The electrode shafts (216a, 216b) may comprise a rounded (218a, 218b) or hemispherical tip, or a ball tip. To provide shielding and/or to facilitate handling of the electrodes, may also comprise optional separate elongate supports (219a, 219b) or jackets, which may increase the cross-sectional diameter or size of the electrodes. In other examples, a single support may be provided and shared by both electrode shafts. The supports (219a, 219b) may have a circular, square or polygonal cross-sectional shape. In some configurations, the two electrode shafts (216a, 216b) each have the same shape, but in other embodiments, the two electrodes may comprise different tip configurations. The electrodes (216a, 216b) may comprise an elongate shaft that may have a length of about 50 mm to about 300 mm, about 75 mm to about 200 mm, or about 100 mm to about 160 cm. The electrode shafts (216a, 216b) may have a square or circular cross section with a diameter in the range of 1 mm to 5 mm, 1.5 mm to 3.5 mm, or 1.5 mm to 2.5 mm. The diameter may be constant along the length of the electrode shaft, or may be larger proximally and have the diameters recited above only in the distal 1, 2, 3, 4 or 5 mm of the electrode. In some variations, the cross-sectional size of the shafts of the electrode shafts (216a, 216b) are larger than the cross-sectional size of the electrode tips (218a, 218b) such that the when the shafts are positioned tightly side-by-side in parallel contact with each other, the desired separation spacing between the electrode tips (218a, 218b) are achieved. In other variations, a separate spacer may be provided between the electrode shafts (216a, 216b) to maintain the desired separation distance. In some further variations, different support or spacer sizes or configurations may be used to manufacture probes with different separation distances.

As noted previously, the electrode shafts (216a, 216b) may comprise a spacing distance or separation gap between about 0.2 mm and about 1.0 mm. In some variations, the electrodes (216a, 216b) may comprise a separation gap between about 0.3 mm and about 0.75 mm. In other variations, the electrodes (216a, 216b) center-to-center distance between a first central longitudinal axis (e.g., through the center or midpoint) of the first electrode (216a) and a second central longitudinal axis of the second electrode (216b) may be between about 0.3 mm and about 2.0 mm. In some other variations, the electrode tips (218a, 218b) may comprise a center-to-center distance about 0.6 mm and about 1.5 mm. To maintain the desired separation gap or spacing distance between the electrodes (216a, 216b), the electrodes (216a, 216b) may be overmolded with a spacing block or in a probe cap, where the ends of the electrodes have a parallel orientation and at the same location relative to the longitudinal axis of the probe (200).

To facilitate manipulation of the probe (200), a handle (220) may be molded around or over, or assembled with the electrodes (216a, 216b). In the embodiment depicted in FIGS. 2A to 2D, the handle (218) comprises multiple assembled components, with a distal probe tip (222), a middle handle (224) and a back cap (226). The components (222, 224, 226) of the handle (220) may comprise a complementary overlapping interfit structures that may be glued, welded, or heat inched together. A friction fit may also be provided so that gluing, welding or heat melting is not required.

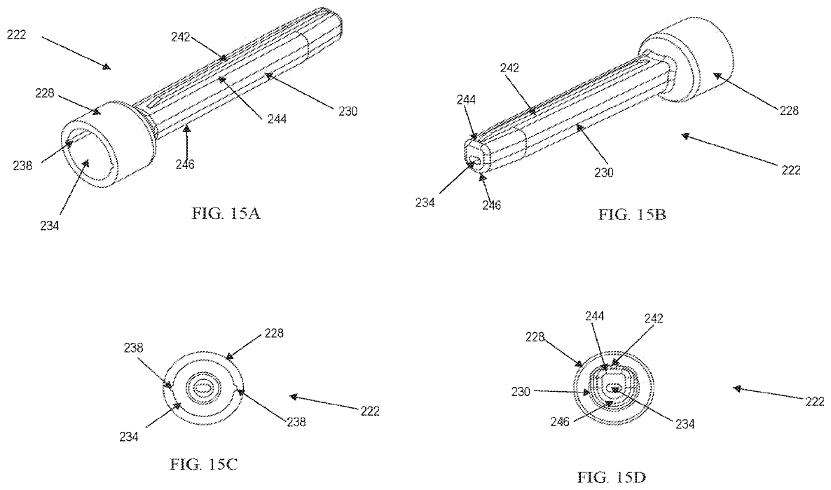

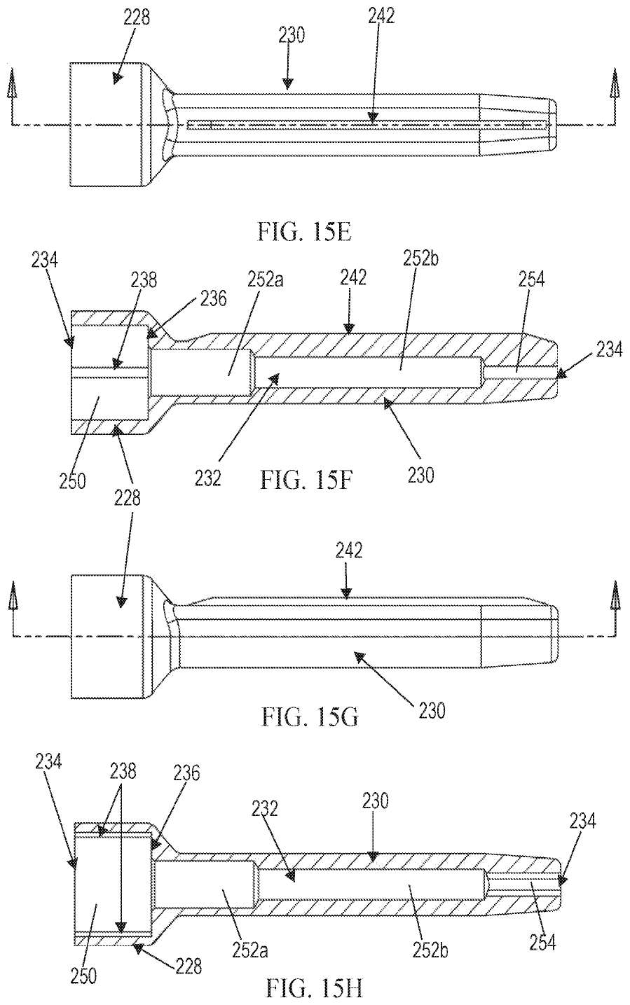

FIGS. 15A to 15H depict various detailed views of the distal probe tip (222). The distal probe tip (222) comprises a proximal connector (228) and a distal shaft (230) and a longitudinal lumen (232) along the length of the probe tip (222). The proximal connector further comprises a proximal opening (234) is sized and configured to receive or form a complementary interfit with the middle handle (224), in addition to the insertion of the electrodes (216a, 216b) during the assembly process. The longitudinal lumen (230) terminates distally at an oblong or rectangular electrode opening (234) from which the two electrodes (216a, 216b) may project out in a side-by-side configuration. Typically, the ratio of the height to width of the opening (234) and portions of the longitudinal lumen (232) will be in a 1:2 ratio, but in other examples, other ratios may be provided, e.g. 1:1, 2:3, or 1:3 for example, or a ratio greater than 1:2, e.g. 1:2.1 to 1:2.3, or 1:1.1 to 1:1.3 or 1:3.1 or 1:3.3. In some variations, the height of the lumen (232) at the distal opening (234) is 0.045″ to 0.055″, or 0.048″ to 0.05″, and the width is 0.09″ to 0.12″, or 0.10″ to 0.11″. The distal shaft (230) may have a length in the range of 1″ to 5″, or 1″ to 3″, or 1.5″ to 2.5″. The length of the distal shaft may be configured to facilitate use in the oral cavity, but in other variations may have a different length for use with other diagnostic EMG procedures, e.g, anal sphincter function, as described elsewhere herein. Although only a single opening is provide in the particular example in FIGS. 15A to 15H, in other variations, two separate distal openings may be provided, rather than a single comprises a longitudinal cross-sectional shape. In still other examples, a tripolar electrode system may be provided, with the three electrodes in a side-by-side-by-side configuration, or a triangular configuration, where each electrode is equally or not equally spaced from the other two electrodes.

Referring still to FIGS. 15A and 15H, the distal probe tip (222) may comprise a proximal orthogonal stop surface (236) spaced distally from the proximal opening (234) to limit the distance of insertion of the middle handle (224). Along the segment of the longitudinal lumen (232) between the opening (234) and the stop surface (238) may also comprise one or more alignment grooves (240), which may form a complementary interfit with corresponding ridges on the middle handle (224). These alignment structures may help to maintain the desired parallel alignment of the electrodes (216a, 216b) during to assembly process, by reducing the risk or incidence of twisting of the wiring or electrodes. In other examples, the relationship of the grooves and ridges may be switched with respect to the probe tip (222) and the middle handle (224), and in still other embodiments, a different number or different type of complementary interfit alignment structures may be provided, such as complementary tapered flange and circular ridge structures configured to attach the middle handle and probe tip, with or without additional bonding or melting to secure the attachment.

In the particular embodiment depicted in FIGS. 2A to 2E, the separation distance or spacing between the electrodes (216a, 216b) are maintained by the outer physical constraint provided by the size and oblong shape of the longitudinal lumen (232) and the distal electrode opening (236). In some clinical uses, the ability of the clinician to position and achieve a specific rotational orientation of the two electrode ends (218a, 218b) may be facilitated by indicia provided on the surface of the probe (220) or indicia from the general shape of the probe (220). In the example depicted in FIGS. 2A to 2E and FIGS. 15A and 15B, the distal shaft (230) may comprise a longitudinal fin or ridge (242), to indicate the side-by-side orientation of the electrodes (216a, 216b) of the each side of the plane intersecting the ridge (242) and the central longitudinal axis of the probe tip (222). The distal shaft (230) also comprises a lower rounded surface (244) and an upper flat surface (246) on which the ridge (242) is located. In some variations, the rounded and flat surfaces (244, 246) may be used to indicate electrode orientation without providing a ridge or outer indicia. In still other examples, printed, raised, or embossed rotational orientation indicia may be provided on the middle handle (224) and/or back cap (226).

The longitudinal lumen may comprise a variety of configurations. In the specific embodiment depicted best in the cross-sectional views at FIGS. 15F and 15H, the longitudinal lumen (232) comprise a proximal receiving section (250) to receive a portion of the middle handle (224) and which is in distal continuity with one or more transition sections (252a, 252b) and a distal section (254) that terminates at the distal opening (234). As described elsewhere, one or more sections of the lumen (232), such as the distal section (254) may be configured with tight tolerances in order to constrain the electrode shafts (216a, 216b) in order to maintain or provide the desired separation spacing between the electrode tips (218a, 218b). In the embodiment depicted in FIGS. 15F and 15H, the transition sections (252a, 252b) have a cross-sectional dimension or size that is smaller than the cross-sectional dimension or size of the proximal receiving section (250), but larger than the cross-sectional dimension or size of the distal section (254). Although each of these sections (250, 252a, 252b, 254) have distinct, stepped transitions between them, other examples, between sections (252a, 252b, and/or 254) the transitions may be more tapered or gradual. Typically, an orthogonal stop surface (236) may be provided at the junction of the proximal receiving section (250) and the transition section(s) (252a, 252b) to facilitate the assembly of the handle (220), but in other examples a stop surface is not required because the a stop surface may be provided on the middle handle portion instead. In some examples, transition section (252a) may have a transverse width or dimension in the range of 0.2″ to 0.3″, or 0.2″ to 0.23″, and transition section 9252b) may have a transverse width or dimension in the ranee of 0.1″ to 0.2″, or 0.13″ to 0.15″.

In other example, the maximum spacing or separation of the electrodes (216a, 216b) may also, or alternatively be constrained together using heat shrink tubing or an outer jacket around the two electrodes. FIG. 2D, for example, depicts distal heat shrink tubing (248) that may be used over the proximal portions of the electrodes (216a, 216b) during assembly. The distal heat shrink tubing may or may not comprise a length such that the heat shrink tubing does not extend out of the distal opening (234) of the probe tip (222), or resides completely outside of the probe tip (222), abutting the distal opening (234), as depicted in FIG. 2C. Heat shrink tubing may also be used to cover any interconnects or soldered and/or crimped components of the probe (200), as described below.

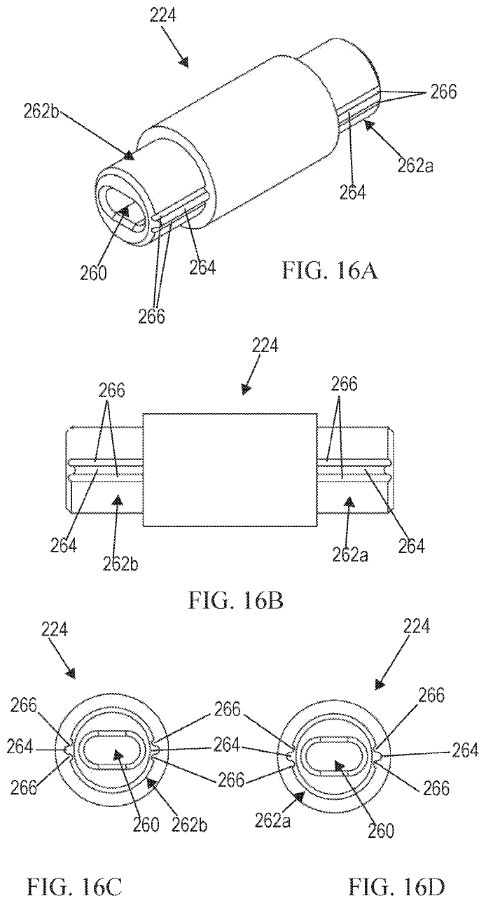

The middle handle (224), depicted in FIGS. 16A to 16D, may comprise a tubular body with longitudinal lumen (260) with a rectangular, oval or oblong cross-sectional shape configured to house the two electrode shafts (216a, 216b), in a side-by-side configuration. The middle handle (224) may comprise a first end section (262a) configured for coupling or insertion into the probe tip (222) and a second end section 9262b) configured for coupling or insertion into the back cap (226). In the particular embodiment depicted in FIGS. 16A to 16D, the first and second end sections may have an identical configuration each end (262a, 262b) may be coupled to either the probe tip (222) or back cap (226) during assembly. As noted previously, each end section (262a, 262b) comprise on or more ridges (264) to limit rotation between the middle handle (224) and the probe tip (222) and/or back cap (226) during assembly, which may facilitate alignment of the electrode tip and spacing. To reduce insertion friction, the middle handle is also provide with grooves (266) immediately adjacent to one or both sides of the ridges (264), optionally without a corresponding ridge provided on the probe tip (222) and/or back cap (226). The presence of the grooves may be provided to offset an increase caused the ridge as a result of increased surface area, and/or may facilitate separation of the middle handle (224) from the mold during injection molding.

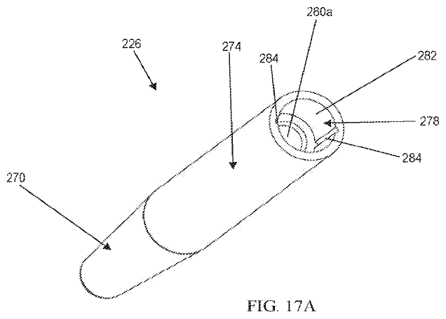

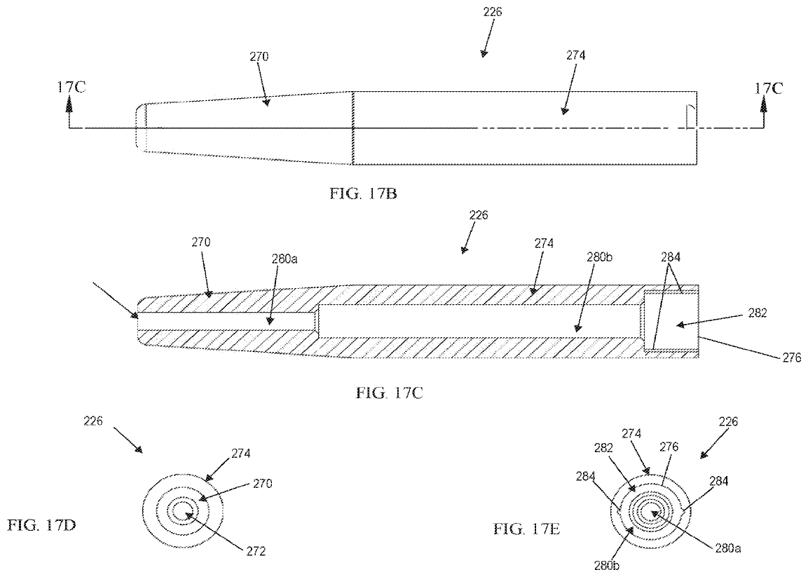

Referring now to FIGS. 17A to 17E, the back cap (226) of the handle (220) comprises a proximal section (270) with a proximal opening (272) and a distal section (274) with distal opening (276) and a longitudinal lumen (278) therebetween. In other variations, addition sections may be provided, or a single body may be provided. The proximal section (270), as depicted is optionally tapered externally from the distal to proximal direction, while the distal section (274) optionally has a constant outer shape or diameter externally. The longitudinal lumen (278) may comprise one or more sections (280a, 280b, and 282), with distinct transitions as shown in FIG. 17C, but in other examples, one or more transitions may be more gradual or the lumen may comprise a single lumen with a gradual taper along its entire length. The transition locations of the lumen sections (280a, 280b, 282) may or may not align with the transition between the proximal and distal sections (270, 274). (As previously described, the distal section (282) of the lumen (278) is configured to mate or receive an end (262b) of the middle handle (224), and may comprise one or more grooves (284) and an optional orthogonal stop surface (286) to facilitate precise the desired rotational orientation and/or fitting of the two components. In some variations, the first section (282a) may have a diameter of 0.1″ to 0.3″, or 0.15″ to 0.2″, and a length of about 1″ to 2″, or 1.1″ to 1.5″. The second section (282b) may have a diameter of 0.2″ to 0.4″, or 0.25″ to 0.35″, and a length of about 2″ to 4″, or 2″ to 3″.

In other variations, instead of a three component assembly for the handle, a two component assembly may be provided with a distal section and a proximal section that are then joined together. For example, the two component assembly may be similar in configuration to the three-component assembly except the middle handle and the back cap may be integrally formed as a single proximal section. In still another embodiment, a first elongate section and a second elongate section that are assembled side-to-side, rather than end-to-end. The two elongate sections may also be interconnected by one or more hinges and clamped around the internal electrode components in a clamshell configuration. Also, one of skill in the art will understand that while the embodiment depicted in the figures comprises a middle handle (224) configured with male ends (262a 262b) to insert into female openings (234, 276) of the probe tip (222) and back cap (226), in other variations the complementary relationship may be reverse, or comprise a different mechanical interfit and/or friction fit interface.

Although the probe embodiments described herein may be configured to with a single multi-wire cable split at its proximal end and directly soldered or crimped to terminals separate or an integrated plug, and split at its distal end and directly soldered crimped to the electrode shafts, in some variations, the plugs and/or electrodes may pre-attached to existing lengths of wire or cable. In some variations, this permits a modular manufacturing process with different plugs for compatibility with different EMG systems or different electrodes to suit specific research purposes. In one example, two DIN 42-802 plugs with pre-attached wires are stripped of a ¼″ of wire jacket and the exposed wires are tinned using RoHS compliant solder. A length of three-conductor cable is cut to a desired length, e.g. 60 to 100 inches, and 2.5″ of the outer jacket of one end of the multi-conductor cable is stripped. 1″ of two exposed conductors is removed and ¼″ each of the wire jacket of the red and black exposed conductors is removed. The two exposed conductors and the unjacketed ground wire are then tinned. A length of heath shrink tubing (HST) is placed over the ground wire, and shrunk to cover the exposed length of the ground wire except for the tinned end. A 3″ length of HST is positioned loosely over the outer jacket of the cable but not heat shrunk yet. Two lengths of HST are then placed over the red and black conductors. The red and black conductors are then soldered to each of the plugs and the corresponding HST is then placed over the soldered regions. A 1″ segment of HST is placed over the ground conductor wired and then the ground conductor wire is soldered and/or crimped to a connector pin, and then the corresponding HST is shrunk covering at least half of the pin connector.

The cable is then threaded through the proximal opening of the back cap of the probe handle, and the distal end of the cable is stripped of 1.5″ of its outer jacket. The ground wire is cut at the termination of the outer jacket, and 2″ of HST are placed over the jacket. The red and black conductor are then stripped of ¼″ of their individual jackets. Switching next to the electrode assembly, a segment of HST is then placed over the side-by-side electrode shafts, approximately 2″ to 2.5″ proximal to the distal tips of the electrodes while confirming that the distal tips are longitudinally aligned, and shrunk tightly around the electrodes. Longitudinal alignment may be facilitated by using an assembly block with a close-ended oblong lumen in which to seat the electrode tips. 1″ of HST are then placed over each of the red and black conductors of the distal end of the cable. The red and black conductors of the cable are then soldered or crimped to the ends of the electrode wires, and then the corresponding HST are shrunk over the connections. The 3″ segment of unshrunk HST, still located over the cable outer jacket, is then repositioned distally over the two shrunk HST at a position 5.5″ to 6″ proximal to the electrode tips, tightly flush against the electrode supports of electrode assembly. The electrode assembly is then inserted through the middle handle component, and then through the distal probe tip component, while confirming that the electrode shafts remain parallel and aligned at the electrode tips. A 1.5″ to 2″ segment of medical grade HST is then cut and placed over the electrode assembly so that the proximal end of the medical grade HST is pushed tightly against probe tip, without leaving any gap therebetween, and then shrunk. Continuity of the electrical connections may be checked with a multi-meter or other electrical test at the end of the assembly process or at one or more timepoints during the assembly process, e.g. after each solder or crimp connection is made.

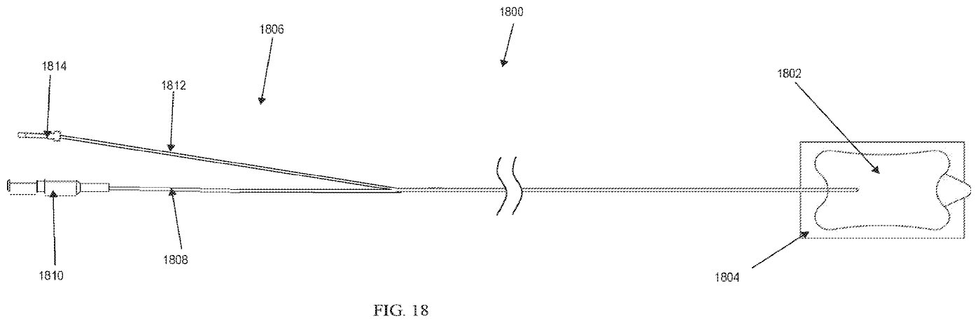

Because of the bipolar configuration of the probe, a standard unipolar adhesive patch ground surface electrode with a single connector plug may or may not be used. Referring to FIG. 18, the ground electrode assembly (1800) may comprise a distal unipolar patch electrode (1802) with a skin adhesive layer (not shown) and a removable release liner (1804) coupled to the skin adhesive layer. The ground electrode assembly (1800) further comprise a split proximal end (1806), with a first proximal end (1808) comprising a connector (1810) or plug for attachment to the ground connector of the EMG system, e.g. a touch proof, single pole connector such as a DIN 42-802, and the a second proximal end (1812) with a connector (1814) to attach to the pin of the electrode assembly, e.g. a female terminal connector that is complementary to the pin. In other examples, a different pair of complementary electrical connectors may be provided for the electrode assembly and ground patch electrode.

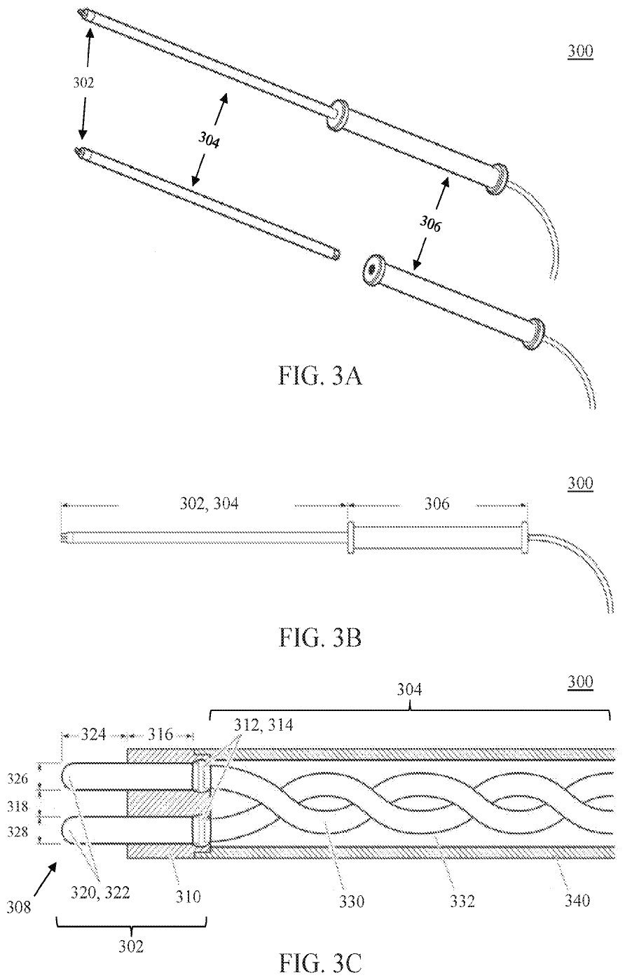

FIGS. 3A-3C are illustrative views of another example of a rigid sensor assembly (300). The sensor assembly (300) may comprise a distal portion (302) (e.g., bipolar or multipolar sensors), intermediate portion (304) (e.g., shall (304)), and a proximal portion (306) (e.g., handle). The proximal portion (306) may be configured as a handle for an operator to grasp and control the sensor assembly (300). The handle may have any suitable length and shape. The intermediate portion (304) and the proximal portion (306) may each comprise a hollow lumen and house therein one or more of an insulated cable (e.g., lead wires), a power cable, a flexible printed circuit, other electronics, and the like. In some variations, the intermediate portion (304) may comprise a rigid shaft, a semi-flexible shaft, and/or comprise portions having combinations thereof. The intermediate portion (304) described herein may be any elongate body suitable for advancement through at least a portion of one or more body lumens and/or cavities. The intermediate portion (304) may be hollow, partially hollow, and/or partially solid. One or more portions of the intermediate portion (304) may be flexible or semi-flexible, one or more portions may be rigid or semi-rigid, and/or one or more portions may be configured to transition between flexible and rigid configurations. Flexible portions of the intermediate portion (304) may allow it to be navigated through tortuous body lumens to reach a desired target site. The intermediate portion (304) described here may be made of any material or combination of materials. For example, the intermediate portion (304) may comprise one or more metals or metal alloys such as nickel titanium alloys, copper-zinc-aluminum-nickel alloys, copper-aluminum-nickel alloys, combinations thereof, and the like, and/or one or more polymers such as silicone, polyvinyl chloride, latex, polyurethane, polyethylene, PTFE, nylon, combinations thereof, and the like.

The intermediate portion (304) may have any suitable dimensions. For example, the intermediate portion (304) may have any suitable length that allows the assembly (300) to be advanced from a point external to the body to a target location. In some variations, the length of the intermediate portion (304) may be between about 1 cm and about 100 cm. The intermediate portion (304) may have any suitable diameter, such as, for example, about 5.7 French, about 6.1 French, about 7 French, about 8.3 French, between about 5 French and about 9 French, between about 5 French and about 7 French, between about 6 French and about 9 French, and the like. The intermediate portion (304) may be removably attached from the proximal portion (306). In some variations, the distal portion (302) may be removably attached to the intermediate portion (304). In other variations, the distal portion (302) may be fixed to the intermediate portion (304).

The distal portion (302) may comprise one or more bipolar or multipolar sensors (308) each comprising a first electrode (320) having a first diameter (326) and a second electrode (322) having a second diameter (328). In some variations, the electrodes (320, 322) may have a spacing distance (318) between each other and a projection length (324) from a surface of the sensor housing portion (310). The housing portion (310) may have a housing length (316). The shape, dimensions, and materials of the bipolar sensor (308) may be the same as those described herein with respect to any of the bipolar sensors disclosed such as bipolar sensors (100, 200, 430, 512). The first and second electrodes (320, 322) may be coupled to respective lead wires (330, 332) through corresponding connectors (312, 314) (e.g., weld points) as described herein.

In some variations, a flexible printed circuit may be disposed in one or more of the intermediate portion (304) and the distal portion (306) of the assembly (300) and coupled to the bipolar or multipolar sensor (308). One or more flexible printed circuits may be configured to electrically connect the bipolar sensor (308) to a controller. Also, in some examples, the probe may be a flexible or malleable probe, or comprise a combination of one or more rigid sections, flexible sections and malleable sections.

E. Probe and Delivery Device

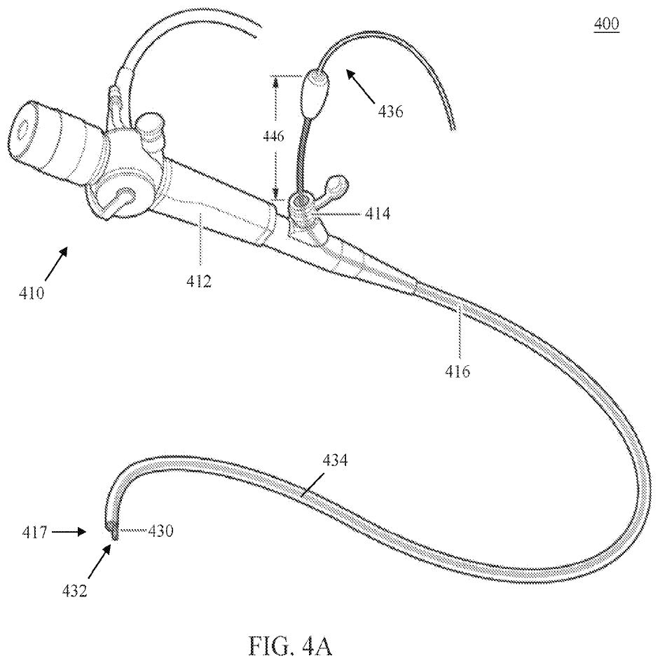

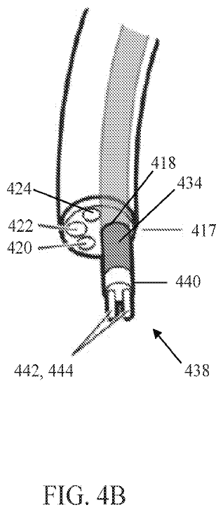

FIGS. 4A-4B are illustrative perspective views of another variation of a sensor system (400) comprising a sensor assembly (430) (e.g., probe) and a delivery device (410) (e.g., access device, visualization device) and configured for one or more of advancement, placement, and visualization of the sensor assembly (430). In some variations, the sensor assembly (430) may be configured to slidably advance through an endoscope (410) to be located topically on membranes in targeted areas of the gastrointestinal (GI) tract to measure EMG activity of muscles associated with GI function such as pharyngeal muscles, a cricopharyngeus, esophageal muscles, a lower esophageal sphincter, a pancreatic sphincter and a bile duct sphincter. In some variations, the sensor assembly may be configured to slidably advance through a catheter of a delivery device to be located topically on membranes in targeted areas of the heart to measure EMG activity of the myocardium. In some variations, the sensor assembly may be configured to pass through an endoscope to be located topically on membranes in targeted urologic areas of the body to measure EMG activity of muscles associated with urologic functions such as a detrusor muscle, a urethral sphincter, a bladder, and the like. In some variations, the delivery device (410) may comprise one or more adult and pediatric flexible endoscopes, laryngoscope, rhino laryngoscope, laryngeal strobe scope, bronchoscope, Zenker's scope, esophagoscope, colonoscope, sigmoidoscope, cystoscope, fiberscope, camera, external light source, imaging sensor, combinations thereof, and the like. The delivery device (430) may be configured to visualize one or more of the sensor probe and anatomic surfaces of a body cavity such as a nasopharynx, oropharynx, hypopharynx, larynx, and esophagus.

In some variations, the delivery device may comprise an optical sensor (e.g., a charged coupled device (CCD) or complementary metal-oxide semiconductor (CMOS) optical sensor) and may be configured to generate an image signal that is transmitted to a display. For example, in some variations, the delivery device may comprise a camera with an image sensor (e.g., a CMOS or CCD array with or without a color filter array and associated processing circuitry). An external light source (e.g., laser, LED, lamp, or the like) may generate light that may be carried by fiber optic cables or one or more LEDs may be configured to provide illumination. For example, a fiberscope comprising a bundle of flexible optical fibers may be configured to receive and propagate light from an external light source. The fiberscope may comprise an image sensor configured to receive reflected light reflected from a body cavity. The image sensor may detect the reflected light and convert it into image signals that may be processed and transmitted for display. The endoscope may have any suitable configuration, for example, it may be a chip-on-the-tip camera endoscope, a three camera endoscope, and the like. The sensor probes may be configured for a forward, side or retro-facing direction.

In some variations, the delivery device (410) may be a catheter for angiography including, transvascular analysis of muscle activity such a myocardium, chordae, cardiac valves including tricuspid, mitral, pulmonic, and aortic valves. In some variations, the sensor assembly may be incorporated into robotic surgical systems, computer navigated surgery systems, and/or minimally invasive surgery systems. This may include using a tool or end effector provided on a robotic arm, catheter, endoscope or minimally invasive diagnostic or surgical device to be positioned on membranes in targeted areas of the gastrointestinal (GI) tract, urinary tract, and oropharyngeal cavity, for example.

The endoscope (410) may comprise a body portion (412) including a port (414) coupled to a catheter (416) having a distal end (417). The sensor assembly (430) may comprise a proximal portion (436) (e.g., handle), a flexible intermediate portion (434), and a distal portion (432). The sensor assembly (430) may be advanced through the port (414) and through the catheter (416) such that the distal portion (432) of the sensor assembly (430) is slidably advanced out of the distal end (417) of the catheter (416). The intermediate portion (434) may be sized to slidably advance within a lumen of the catheter (416). The proximal portion (436) may comprise a handle, insulated lead wires, and the like. In some variations, the proximal portion (436) may comprise a length in the range of between about 1 cm and about 3 m. The proximal portion (436) may be separated from the port (414) by a predetermined distance (446). In some variations, the distance from the port (414) to a handle may be in the range of between about 10 cm and about 30 cm.

FIG. 4B is a perspective view of the distal end (417) of the catheter (416) and a bipolar sensor (438) of the sensor assembly (430). The distal end (417) of the catheter (416) may comprise a first lumen (418), second lumen (420), third lumen (422), and fourth lumen (424). In some variations, the first lumen (418) may be configured as a sensor assembly lumen for a sensor assembly (430) to slidably advance through. The second lumen (420) may be configured as an optical sensor lumen for an optical sensor (not shown) to be disposed in. A third lumen (422) may be configured as a light source lumen for a light source (not shown) to be disposed in. A fourth lumen (424) may be configured as a guidewire lumen for a guidewire (not shown) to slidably advance through. The number of lumens in the catheter (430) is not particularly limited and may include one, two, three, four, and five or more lumens.

The catheter (416) and intermediate portion (434) described herein may comprise any elongate body suitable for advancement through at least a portion of body lumen. The catheter (416) and intermediate portion (434) may be hollow, partially hollow, and/or partially solid. One or more portions of the catheter (416) and intermediate portion (434) may be flexible or semi-flexible, one or more portions may be rigid or semi-rigid, and/or one or more portions of the catheter (416) and intermediate portion (434) may be changed between flexible and rigid configurations. Flexible portions of the catheter (416) and intermediate portion (434) may allow the catheter (416) and intermediate portion (434) to be navigated (e.g., steerable) through tortuous body lumens to reach a desired target site. The catheter (416) and intermediate portion (434) described here may be made of any material or combination of materials. For example, the catheter (416) and intermediate portion (434) may comprise one or more metals or metal alloys (e.g., e.g., nickel titanium alloys, copper-zinc-aluminum-nickel alloys, copper-aluminum-nickel alloys, combinations thereof, and the like) and/or one or more polymers (e.g., silicone, polyvinyl chloride, latex, polyurethane, polyethylene, PTFE, nylon, combinations thereof, and the like) as described herein.

Catheter (416) and intermediate portion (434) may have any suitable dimensions. For example, catheter (416) and intermediate portion (434) may have any suitable length that allows those components to be advanced from a point external to the body to a target location. In some variations, the catheter (416) may have a length in the range of between about 20 cm and about 200 cm. Catheter (416) and intermediate portion (434) may have any suitable diameter, such as, for example, about 5.7 French, about 6.1 French, about 7 French, about 8.3 French, between about 5 French and about 9 French, between about 5 French and about 7 French, between about 6 French and about 9 French, and the like. The intermediate portion (434) may be removably attached to the proximal portion (436). In some variations, the bipolar sensor (438) may be removably attached to the intermediate portion (434).

The bipolar or multipolar sensor (438) may be disposed within a distal portion (432) of the sensor assembly (430). The bipolar sensor (438) may comprise a sensor housing (440) coupled to a first electrode (442) and a second electrode (444). The shape, dimensions, and materials of the bipolar sensor (438) may be the same as those described herein with respect to any of the bipolar sensors disclosed such as bipolar sensors (100, 200, 312, 512).

### III. Methods

Also described here are methods for non-invasively generating an EMG signal corresponding to muscle tissue using the systems and devices described herein. The methods described here may permit EMG data to be gathered from difficult to reach tissue and measure locations such as moist body cavities and organ systems. This may have numerous benefits, such as permitting evaluation and diagnosis of a range of neuromuscular conditions. Conventional EMG techniques may be inadequate for generating EMG data in many body cavities and organ systems. For example, NEMG in body cavities and organ systems has hinted clinical use due to a higher risk of damage to tissue. SEMG may be inadequate for evaluating neuromuscular function due to its lower spatial resolution and contamination from one or more electrical, mechanical, and movement artifacts. Furthermore, SEMG systems having adhesive surface electrodes may not be appropriate for organ systems and moist body cavities.

Generally, the methods described here include advancing a sensor probe into a body cavity to contact a tissue surface, and receive signal data without penetrating or piercing the intact tissue surface. The signal data may be processed and used to generate electromyography data. It should be appreciated that any of the systems and devices described herein may be used in the methods described herein.

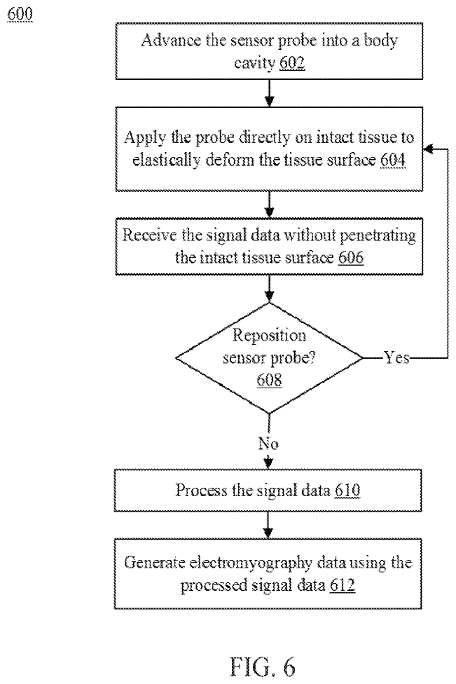

FIG. 6 is a flowchart that generally describes a method of using a sensor probe (600). The process (600) may begin by advancing a sensor probe into a body cavity (602), organ system, or anatomical structure. One or more sensors of the probe may be configured in a bipolar or multipolar configuration. The probe may be rigid or flexible. For example, a rigid probe may be straight, curved, or have one or more straight portions and curved portions. In some variations, the (rigid or flexible) probe may be used in conjunction with a separate delivery device, as described in detail herein.

The probe may be applied directly on intact tissue to elastically deform the tissue surface (604). For example, the probe may atraumatically contact the surface of target tissue and/or a membrane overlying the target tissue. Neither the sensors nor probe penetrates or pierces the intact target tissue. In some variations, the probe may contact the target tissue and/or membrane at an angle between about 60 degrees and about 120 degrees with respect to a surface of the target tissue and/or membrane in contact with the probe. For example, the probe may be perpendicular to the target tissue and/or membrane. In some variations, the probe may contact the target tissue at a midpoint of the muscle to avoid tendinous insertions.

Signal data may be received by the sensors of the probe without penetrating the intact tissue surface (606). For example, the probe may remain in continuous contact with the intact tissue as electrical activity of the target tissue is received by the probe. The tissue surface may be maintained in an unbroken state while applying the one or more sensors of the probe directly on the tissue surface. A determination whether to reposition. the sensor probe (608) may be performed. If so, the probe may be moved and steps 604 and 606 repeated. The signal data received by the sensor probe may be processed (610). For example, the electrical activity signal data received by the probe may be amplified by an amplifier of a controller and/or undergo additional filtering in real-time as the data is being received.

Electromyography data may be generated using the processed signal data (612). For example, the processed signal data may be compared against a threshold (e.g., benchmark activity level) to generate EMG data. In some variations, the EMG data may be generated and displayed to a user in real-time to permit the user to determine if the sensor probe should be repositioned. The received and/or processed signal data and generated EMG data may be stored in memory locally, on another device, and/or over a network (e.g., cloud storage, remote server). The sensor probe may be removed from the target tissue and retracted away from the patient.

The systems, devices, and methods described here may be used throughout the body to generate EMG data and permit evaluation of neuromuscular function and/or diagnosis of neuromuscular conditions, examples of which are described in more detail herein.

A. Transoral EMG Example 1

Conventionally, usage of NEMG in a pharynx and/or tongue is difficult to perform because an awake patient may suffer from one or more of insertion pain, gag reflex, procedure fear, local trauma, and bleeding. Any of these factors may limit needle electrode placement in the transoral cavity. General anesthesia may be used to reduce some of these issues, but the use of anesthesia adds complexity, cost, and other possible complications to the procedure. In some variations, a sensor assembly may comprise a transoral (e.g., airway) probe configured to contact the mucosa and treasure electrical activity of one or more of the underlying muscle tissues such as a palatopharyngeus, palatoglossus, musculus uvulae, vocal cord muscles, intrinsic tongue muscle, genioglossus, cricopharyngeus, tensor veli palatini, levator veli palatini, interarytenoideus, cricoarytenoids, cricothyroid, constrictor muscles, upper esophageal sphincter, lower esophageal sphincter, gastroduodenal sphincter, sphincter of Oddi, gastric, and paraspinal, as described in detail herein.

In variations where a rigid sensor probe is used transorally, the rigid sensor probe may be inserted through a patient's mouth and advanced toward one or more muscles to be measured such as in the nasopharynx, oropharynx, hypopharynx, larynx, esophagus, and GI tract. In other variations, a rigid sensor probe may be used with a visualization device such as a rigid straight laryngoscope, rigid laryngeal strobe scope, rigid curved laryngoscope, rigid bronchoscope, rigid esophagoscope, and rigid diverticuloscope to measure.

In variations where a flexible sensor probe is used transorally, the flexible sensor probe may be inserted through a patient's mouth and advanced toward one or more muscles to be measured. In some other variations, a flexible sensor probe may be used with a visualization device such as a flexible fiber optic rhinolaryngoscope, flexible fiber optic bronchoscope, flexible fiber optic esophagoscope. For example, the flexible sensor probe may be slidably advanced through a lumen of the flexible visualization device.

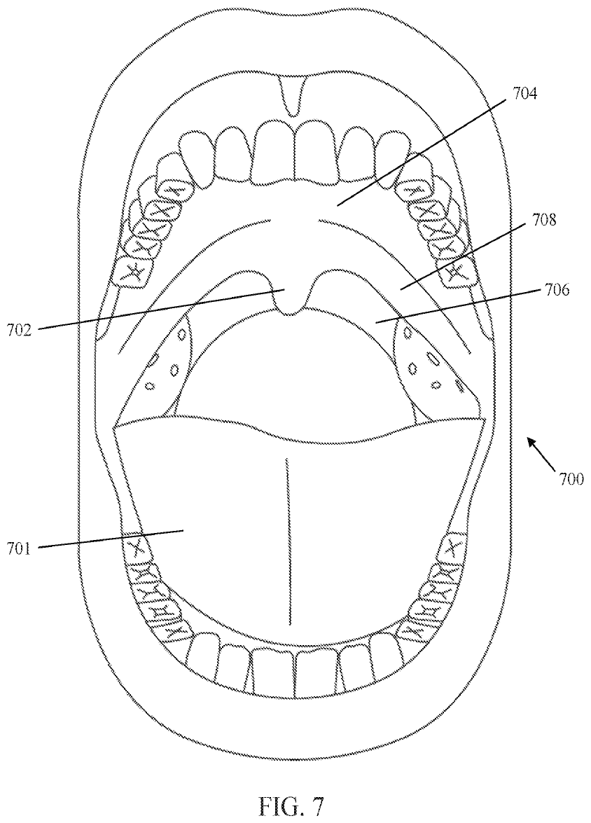

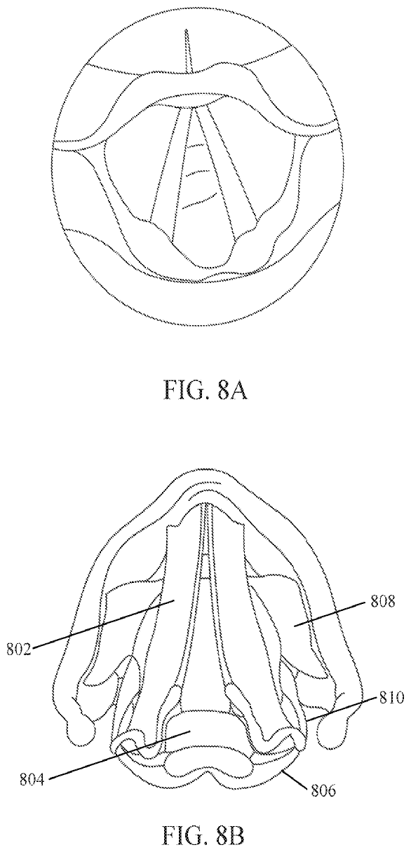

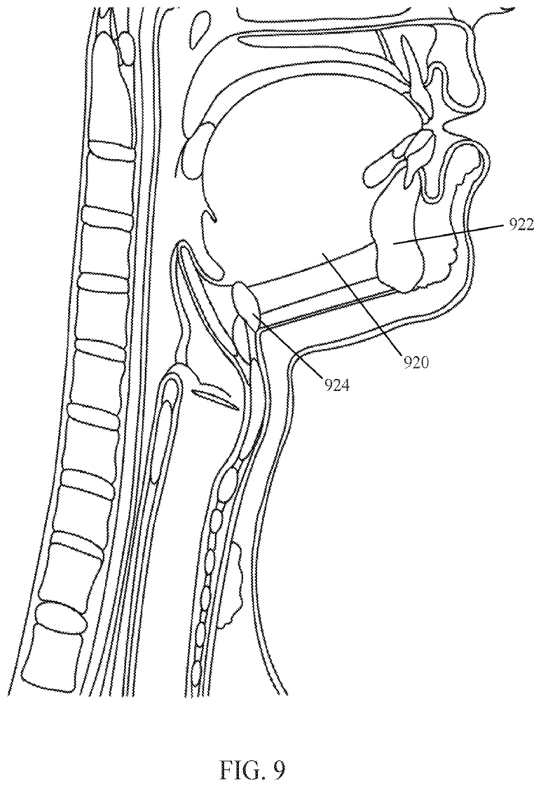

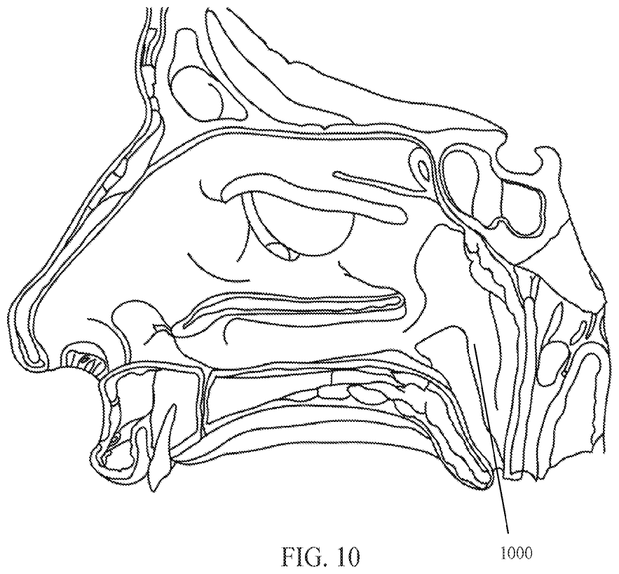

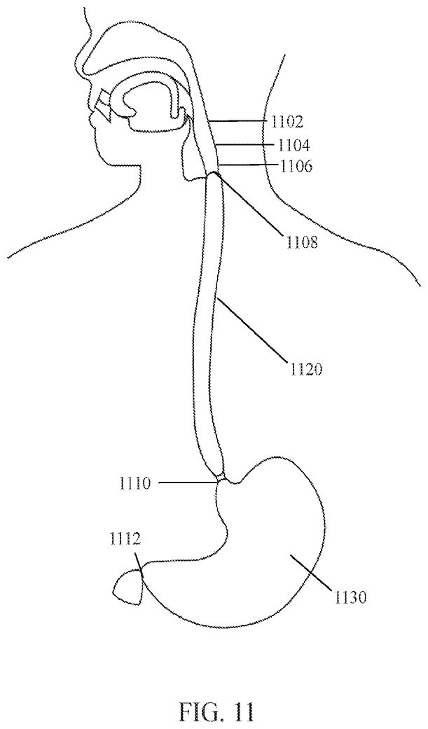

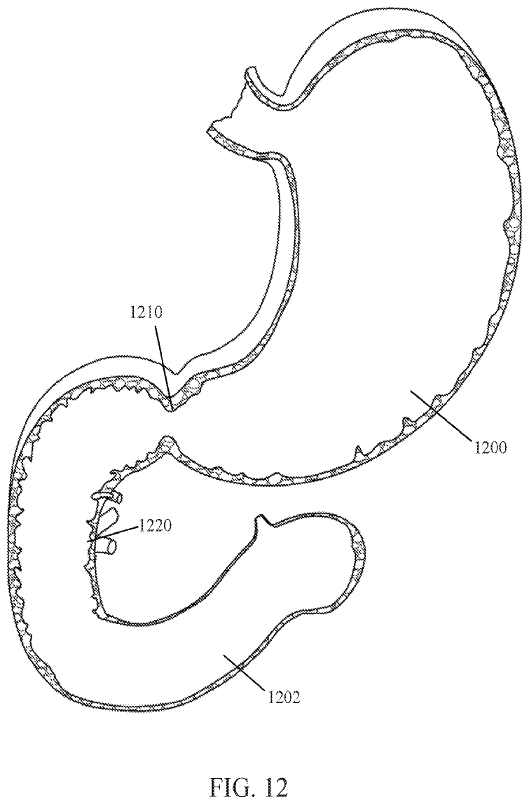

To illustrate some of the methods of using a sensor probe, some of the muscles that may be sensed by the probes described herein are illustrated with respect to the FIGS. 7-12. For example, FIG. 7 is a frontal surface view of the oropharynx (700), FIG. 8A is an axial surface view of the larynx (800), FIG. 8B is an axial view of the larynx musculature, FIG. 9 is a sagittal cross-sectional view of the oropharynx, hypopharynx and larynx, and FIG. 10 is a cross-sectional view of the nasopharynx (1000). FIG. 11 is a cross-sectional view of a portion of the upper gastrointestinal tract and FIG. 12 is a cross-sectional view of the stomach (1200) and duodenum (1202).

For example, the probe may be configured to contact the mucosa overlying a side of the musculus uvulae (702) projecting (e.g., descending) from a posterior edge of the middle of the soft palate (704). The probe may be configured to contact the mucosa overlying one or more of the posterior fasciculus and anterior fasciculus of the palatopharyngeus (706). The probe may be configured to contact the mucosa overlying one or more of the four intrinsic muscles that extend along a length of the tongue including the superior longitudinal muscle that extends along an upper surface of the tongue, the inferior longitudinal muscle that extends along a side of the tongue (701), the vertical muscle located along the middle of the tongue (701) (and which joins the superior and inferior and longitudinal muscles), and the transverse muscle that divides the tongue (701) at the middle. The probe may be configured to contact the mucosa overlying one or more of the genioglossus (920) arising from the mental spine of the mandible (922) and the hyoid (924), and the palatoglossus (708) arising from the palatine aponeurosis of the soft palate (704).

The probe may be configured to contact the mucosa overlying the outer circular layer of the pharynx (902), including one or more of the superior (1102), middle (1104), and inferior (906) constrictor muscles. The superior constrictor muscle (1102) couples to the medial pterygoid plate, the pterygomandibular raphè, and the alveolar process. The middle constrictor muscle (1104) extends from the hyoid. The inferior constrictor muscle (1106) couples to the cricoid and thyroid cartilage. The probe may be configured to contact the mucosa overlying the thyroarytenoid muscle (e.g., vocalis) (802) coupled between the inner surface of the thyroid cartilage and the anterior surface of the arytenoid cartilage.

The probe may be configured to contact the mucosa overlying one or more of the tensor veli palatini muscle and levator veli palatini muscle where the tensor veli palatini is anterior-lateral to the levator veli palatini muscle. The tensor veli palatini muscle is coupled to the medial pterygoid plate of the sphenoid bone and the levator veli palatini muscle is coupled to the temporal bone. The probe may be configured to contact the mucosa overlying the inferior pharyngeal constrictor (e.g., cricopharyngeus) that arises from the sides of the cricoid and thyroid cartilage. The probe may be configured to contact the mucosa overlying the interarytenoideus (804) located in the posterior larynx. The probe may be configured to contact the mucosa overlying the cricoarytenoid muscle (e.g., posterior and lateral cricoarytenoid muscle) (806) located between the cricoid cartilage and arytenoid cartilage. The probe may be configured to contact the mucosa overlying the cricothyroid muscle (808) of the larynx. The cricothyroid muscle (808) is coupled to the anterolateral aspect of the cricoid and the inferior cornu and the lower lamina of the thyroid cartilage. The probe may be configured to contact the mucosa overlying the cricoarytenoid muscles (e.g., posterior and lateral cricoarytenoid muscle) (810) of the larynx that connects the cricoid cartilage and arytenoid cartilage.

B. Transnasal EMG Example 2

In some variations, a sensor assembly may comprise a transnasal probe configured to contact and measure electrical activity of one or more muscle tissues such as the Eustachian tube (1002) that links the nasopharynx to the middle ear, superior constrictor (1004), and cervical spine (1006). In variations where a rigid sensor probe is used transnasally, the rigid sensor probe may be inserted through a patient's nostril and advanced toward one or more nasopharynx muscles to be measured. In other variations, a rigid sensor probe may be used with a visualization device such as a rigid endoscope to measure one or more nasopharynx muscles.

In variations where a flexible sensor probe is used transnasally, the flexible sensor probe may be inserted through a patient's nostril and advanced toward one or more nasopharynx muscles to be measured. In some other variations, a flexible sensor probe may be used with a visualization device such as a flexible fiber optic rhinolaryngoscope, flexible fiber optic bronchoscope, flexible fiber optic esophagoscope, and flexible gastroduodenoscope to measure one or more tissue surfaces including the nasopharynx, oropharynx, hypopharynx, larynx, esophagus (as described in detail herein), and GI tract. For example, the flexible sensor probe may be slidably advanced through a lumen of the flexible visualization device.

For example, the probe may be configured to contact the mucosa overlying one or more of the upper esophageal sphincter (UES) (e.g., cricopharyngeal part of the inferior pharyngeal constrictor) (1108) that surrounds an upper portion of the esophagus (1120) and lower esophageal sphincter (LES) (e.g., gastroesophageal sphincter) (1110) that surrounds the lower part of the esophagus (1120) at the junction between the esophagus (1120) and the stomach (1130, 1200). The probe may be configured to contact the mucosa overlying one or more of the gastroduodenal sphincter (e.g., pyloric sphincter) (1112, 1210) surrounding the gastroduodenal junction and the sphincter of Oddi (1220) located between the pancreas and duodenum (1202).

C. Gastrointestinal EMG Example 3

A conventional NEMG needle inserted into an upper and/or lower GI tract may increase the risk of perforation of the viscus and may lead to catastrophic complications related to viscus perforation of the esophagus, duodenum, gallbladder, pancreas, small bowel, large bowel, sigmoid colon, and rectum. In some variations, a sensor assembly may comprise a gastrointestinal probe configured to contact the mucosa overlying muscle tissue such as a lower esophageal sphincter, gastroduodenal juncture, gall bladder, pancreas, small and large intestine sigmoid colon, and rectum. The sensor assembly may comprise one or more flexible and rigid GI endoscopes. The gastrointestinal probe may be disposed in the endoscope using an endoscope port. One or more sensors of the GI probe may be configured to be located on a mucosal surface of the GI tract. In some variations, a distal end of a flexible GI probe may be oriented to be adjacent to a submucosal muscle of interest. For example, the probe may be configured to contact one or more of the lower esophageal sphincter, gastroduodenal sphincter, sphincter of Oddi and gastric tissue, as described in detail herein. In some variations, a rigid probe may slidably advance through a rigid scope such as a rigid esophagoscope, sigmoidoscope, or other operative rigid endoscope.

The EMG data generated from the gastrointestinal probe signal data may be used, in adult and pediatric populations, to evaluate one or more of dysphagia (including the pharyngeal muscles), cricopharyngeus, esophageal muscles, lower esophageal sphincter, pancreatic sphincter, bile duct sphincter, anal sphincter, Ogilvie syndrome, gastrointestinal neoplasm, gallstone ileus, intestinal adhesions causing obstruction, volvulus, intussusception, amyotrophic lateral sclerosis, Brown-Sequard syndrome, central cord syndrome, multiple sclerosis, Parkinson's disease, spina bifida, spinal cord injury, lesions and aging, and diabetes mellitus.

D. Orbital EMG Example 4

Conventionally, usage of NEMG in and around the eye is difficult to perform. For example, an awake patient may suffer from one or more of insertion pain, procedure fear, local trauma, and bleeding. During the procedure, the needle may need to be repositioned, making patient tolerance and diagnostic compliance even more difficult. A needle electrode further carries a higher risk of intraorbital complications that may lead to double vision, infections, hemorrhage, and/or blindness. General anesthesia may be used to reduce some of these issues, but the use of anesthesia adds complexity, cost, and other possible complications to the procedure. As a result, orbital NEMG is typically limited to specific operating room nerve monitoring conditions.

In some variations, a sensor assembly may comprise an orbital probe configured to contact muscle tissue such as one or more eye muscles such as a medial rectus, lateral rectus, superior oblique, inferior rectus, inferior oblique, and superior rectus. One or more sensors may be configured to be located topically on the eye to measure EMG activity of the extraocular muscles through the conjunctival membrane. In some variations, topical anesthesia may be applied to the conjunctiva and the orbital probe may be subsequently placed directly on the conjunctiva of the overlying muscle. The EMG data generated using the orbital probe signal data may be used to evaluate one or more of Battaglia Neri syndrome, dermatomyositis, diabetic neuropathy, encephalitis, meningitis, multiple sclerosis, myasthenia gravis, Parkinson's disease, subacute sclerosing pan encephalitis, tumor infiltration into muscle, Grave's disease, accommodative esotropia, Brown syndrome, strabismus, convergence insufficiency, dissociated vertical deviation, Duane syndrome, esotropia, exotropia, oculomotor apraxia, abnormal head position, conjugate gaze palsies, internuclear ophthalmoplegia, microvascular cranial nerve palsy, monocular elevation deficiency (double elevator palsy), palsies of the cranial nerves that control eye movement, progressive supranuclear palsy, and pseudo strabismus.

E. Endovascular EMG Example 5

In some variations, a sensor assembly may comprise an endovascular probe configured to contact muscle tissue such as myocardium, aortic, mitral, pulmonic, tricuspid valves, and all endothelially lined vascular surfaces where adjacent muscle is anatomically known. One or more sensors may be configured to be located on an endothelial surface that anatomically correlates to a muscle such as a myocardium, heart valves, and chordae tendineae to measure EMG activity of those muscles. The EMG data generated using the endovascular probe signal data may be used to evaluate one or more of transient or chronic autonomic dysfunction related to cerebrovascular disease, myocardial infarction or areas of ischemic muscle tissue, valvular disease and muscle function, left ventricular dysfunction arrhythmia, degenerative brain disorders present with myocardial disease, degenerative neurological conditions, cardiomyopathy, neuromuscular disorders, mitochondrial myopathies, dystrophies, cardiac conduction defects, and renovascular disease. Conventional NEMG needle insertion may increase the risk of perforation of the viscus and may lead to catastrophic complications in the myocardium and vascular system.

F. Orthopedic EMG Example 6

In some variations, a sensor assembly may comprise an orthopedic probe configured to contact muscle directly during surgery in all open muscle operations or via a rigid probe within a synovial cavity of a joint. The sensor assembly may comprise a rigid endoscope placed within the joint during surgical procedures of the joint. One or more sensors may be configured to be located on muscles that traverse through or near a synovial surface such as the shoulder, hip, knee, elbow, ankle, and the like. The EMG data generated from the orthopedic probe signal data may be used, in adult and pediatric populations, to evaluate one or more of muscle activity in open and endoscopic joint surgery, shoulder girdle muscle assessment, hip and knee joint muscle assessment, and any body joint accessible via an open or endoscopic technique.

G. Urologic EMG Example 7

In some variations, a sensor assembly may comprise a urologic probe configured to contact muscle tissue such as one or more of a urethra, urethral sphincter, and bladder. The sensor assembly may comprise one or more flexible and rigid endoscopes (e.g., cystoscope) advanced using a urethra or suprapubic approach. Conventional NEMG needle insertion may increase the risk of perforation of the viscus and may lead to catastrophic complications in one or more of the urethra and bladder. The EMG data generated from the urologic probe signal data may be used, in adult and pediatric populations, to evaluate one or more of a bladder, spermatic cord, ureteropelvic junction, ureter, ureterovesical junction, prostate, testis, and epididymis conditions. For example, bladder conditions may include overactive bladder, interstitial cystitis, post radiation muscle injury bladder, internal and external urethral sphincter, and bladder neck contracture. Spermatic cord conditions may include pain syndromes associated with cremasteric muscle spasms, varicocele associated with infertility and pain syndromes, and congenital malformations (prenatal in utero-transvaginal). Ureteropelvic junction conditions may include ureteropelvic junction (UPJ) obstruction (e.g., scar, cross vessel, overactive muscles). Ureter conditions may include mega ureter (e.g., functional, scar, obstruction), ureterectasis (e.g., functional, obstruction), and hydronephrosis (e.g., functional, obstruction). Ureterovesical junction (UVJ) conditions may include vesicoureteral reflux (VUR), overactive bladder (OAB), neurogenic bladder (with or without spinal cord injury), chronic obstructive uropathy, detrusor sphincter dyssynergia, urethral stricture (to check muscle/scar function versus just scar function), and interstitial cystitis. Prostate conditions may include prostatodynia and/or pain syndromes. Testis conditions may include cryptorchidism retractile testis (e.g., overactive cremasteric muscle, true cryptorchid), and verumontanum/ejaculatory duct opening (e.g., obstruction, functional). Epididymis conditions may include epidydimal dilation (e.g., obstruction, functional). Vas Deferens conditions may include vassal immobility syndromes.

H. Tympanic and Transtympanic EMG Example 8

In some variations, a sensor assembly may comprise a tympanic and/or transtympanic probe configured to be contact muscle tissue such as one or more of a stapedius, including round and oval window, cochlea, and labyrinth. The probe may be configured to access the middle ear. In some variations, a probe in contact with the tympanic membrane may permit evaluation of one or more of the tensor tympani, the oval and round window, facial nerve, cochlea, and labyrinth.

In some variations, the probe may be configured to intraoperatively monitor inner ear function for all middle ear, middle fossa, and neurosurgery where monitoring of the vestibular, cochlear, and/or facial nerve may be performed, such as with acoustic neuroma surgery and inner ear surgery. The EMG data generated from the tympanic/transtympanic probe signal data may be used, in adult and pediatric populations, to evaluate one or more of vertigo including vestibular neuronitis, vestibular migraine, Meniere's disease, BPPV, tinnitus, hearing loss from aging, noise, trauma, autoimmune disease, vascular disease, sudden sensorineural hearing loss, ototoxicity, vestibulotoxicity, trauma, and fractures of the temporal bone and skull.

### IV. Rigid EMG Probe Clinical Testing Example 9

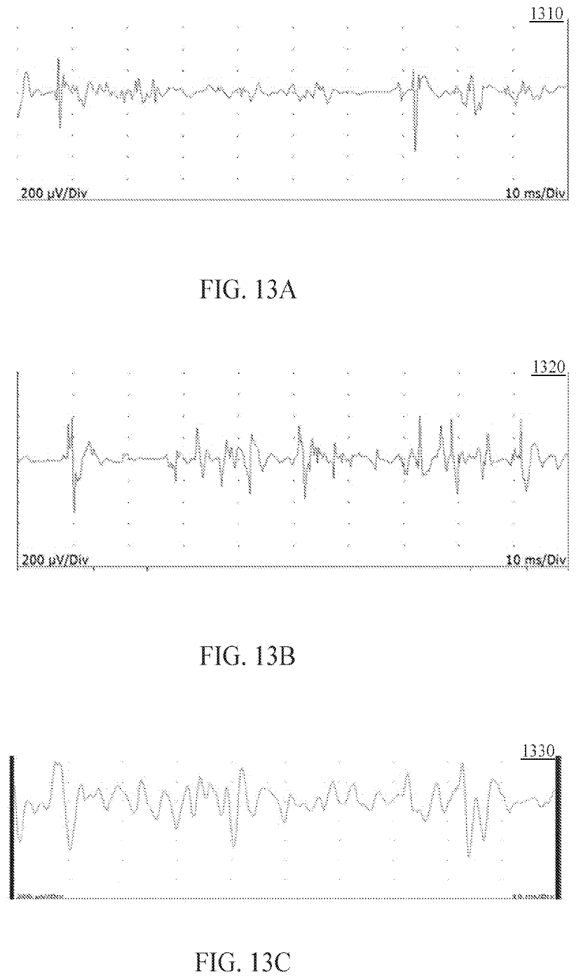

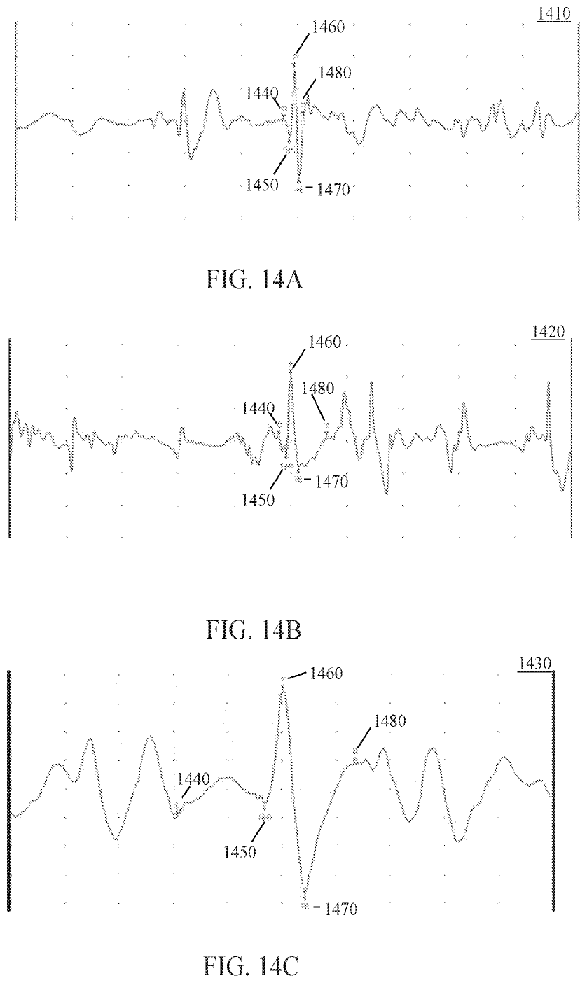

As described herein, EMG data may be generated using the sensor probe assemblies as described herein. For example, FIGS. 13A and 14A are graphs of EMG data (1310, 1410) generated using the systems and methods described herein as applied to a right palatoglossus muscle. FIGS. 13B and 14B are comparative graphs of NEMG data (1320, 1420) of the right palatoglossus muscle of the same patient using a conventional needle electrode. FIGS. 13C and 14C are graphs of SEMG data (1330, 1430) of a right first dorsal interosseous muscle of the same patient using a conventional surface electrode. It should be appreciated that a surface electrode is inadequate to record electrical activity of the palatoglossus muscle due to the moisture (e.g., mucosal lining) between the surface electrode and palatoglossus muscle. Therefore, for the sake of explanation, an external surface SEMG recording of the right first dorsal interosseous muscle (1330, 1430) is provided for comparison with FIGS. 13A-13B and 14A-14B.

As described herein and shown in FIG. 13A and FIG. 13C, the EMG data (1310) generated using the sensor assemblies as described herein has a higher resolution (e.g., detail) than the EMG data (1330) generated using a surface electrode. In contrast, EMG data generated by the sensor assemblies (1310) as described herein and a needle electrode (1320) have similar EMG signal quality due to the close proximity of their sensor electrodes to a motor unit potential of the target tissue. Each of the EMG graphs in FIGS. 13A-13C have a gain and sweep speed set to, respectively, a vertical division of 200 μV/division and a horizontal division of 10 ms/division.

FIGS. 14A-14C correspond to the EMG graphs of respective FIGS. 13A-13C having a shorter timescale such that they depict individual motor unit action potentials (MUAPs). In particular, each of the EMG graphs (1410, 1420, 1430) in FIGS. 14A-14C have a gain and sweep speed set to, respectively, a vertical division of 200 uV/division and a horizontal division of 4 ms/division. A MUAP may have a frequency of between about 30 Hz and about 10 KHz. Each MUAP may comprise a set of characteristics including rise time, amplitude, duration, number of turns, area, phases, thickness, and size index. In particular, each MUAP may comprise a set of points including onset (O—1440), spike onset (Sp—1450), peak (P—1460), peak end (PE—1470), and end (E—1480). MUAP characteristics may be calculated from EMG data in a manual, semi-automated, or automated manner. Table 1 lists a set of MUAP characteristics corresponding to FIGS. 13-14.

### Table 1

TABLE 1

| | Rise | Amp | Dur | Area | | | | Size |
|---|---|---|---|---|---|---|---|---|
| Muscle | (ms) | (μV) | (ms) | (mVms) | Phases | Turns | Thickness | index |
| Palatoglossus | 0.34 | 661.77 | 1.39 | 0.18 | 3 | 3 | 0.28 | −0.08 |
| TM-EMG | | | | | | | | |
| Palatoglossus - | 0.30 | 561.98 | 3.39 | 0.47 | 3 | 3 | 0.84 | 0.34 |
| Needle | | | | | | | | |
| Dorsal | 1.34 | 1421.36 | 13.17 | 2.64 | 4 | 4 | 1.86 | 2.16 |
| Interosseous - | | | | | | | | |
| Surface | | | | | | | | |

Rise time is the duration of a rapid positive-negative deflection (e.g., from spike onset SpO to peak P) and may correspond to a distance of muscle fibers to the electrode. For example, a shorter rise time corresponds to a shorter distance between the muscle fibers and the electrode. Duration may correspond to the time from initial deflection to a final return to baseline (e.g., from onset O to end E). For example, normal MUAPs may have a duration between about 10 ms to about 13 ms, although this range may vary among different muscles. Duration may also correspond to an area of the motor unit. Amplitude may correspond to a maximal peak-to-peak amplitude of a main spike of the MUAP (e.g., from peak P to peak end PE). Amplitude generally corresponds to the action potentials of a set of muscle fibers within the motor unit that are in close proximity to the portion of the electrode in contact with tissue. Phase may correspond to the number of times the action potential crosses the baseline plus one. For example, the MUAP may be monophasic, biphasic, triphasic, or polyphasic. Normal potentials may have three or four phases. A turn may correspond to a potential reversal greater than about 50 μV that does not cross the baseline.

The sensor assemblies as described herein may be configured to elastically deform the tissue surface without penetrating or piercing the intact tissue surface (e.g., intact epithelial surface) while receiving signal data corresponding to EMG data of a motor unit action potential having a rise time of less than about 500 μs. For example, the sensor assemblies as described herein may be configured to receive signal data corresponding to EMG data of a motor unit action potential having a rise time of less than about 300 or 400 μs while maintaining the tissue surface in an intact or unbroken state, e.g. without requiring needle puncture or anatomical dissection to achieve access to the target musculatore. In some variations, the sensor probes as described herein may be repositioned when EMG data indicates motor unit action potential rise times above 500 μs. Surface electrodes used for SEMG are not sensitive enough to generate EMG data with rise times of less than about 500 μs due to the large surface area of the surface electrode.

### V. Rigid EMG Probe Manufacturing Example 10

In one exemplary process for manufacturing an exemplary rigid EMG bipolar probe, a multi-wire cable is provided or cut to a length to a desired length, e.g. 84 inches, or 50 inches to 200 inches. The cable is preferably an RF-shielded cable comprising three coated wires. The outer jacket of each end of the cable and the three wires are stripped, and two wires are soldered, crimped, or otherwise attached to a plug end for insertion or coupling to terminals of a signal amplifier and/or EMG recording equipment.

### VI. Rigid EMG Probe Clinical Study Example 11

In another example, the exemplary probes as described herein may be used to perform transmembrane electromyography (TM-EMG) as a non-invasive EMG technique for detecting and/or assessing neuromuscular (NM) impairment or function. In healthy volunteers or patients with suspected neuromuscular or structural disease, including but not limited to obstructive sleep apnea (OSA) and other diseases that may involve the oropharynx, characteristics motor unit potentials obtained using conventional needle EMG (NEMG) can be compared to assessment using TM-EMG, or with TM-EMG alone. TM-EMG may also be used to assess or confirm that a NM disturbance of oropharyngeal striated muscles in persons with OSA can be elicited using the probes described herein.

Study recruitment will occur at a tertiary referral center which focuses on diseases of the head, neck, brain and spine. All EMG testing procedures will be performed in the clinic under the supervision of an otolaryngologist who will obtain the EMG tracings. In-Laboratory polysomnogram (PSG) studies will be performed at a comprehensive and ACHC accredited sleep diagnostic facility.

The study design will be a prospective, cohort, pilot study with blinded data analysis and two physician testers to assess intertester reliability and device usability. Volunteers will include healthy adults, participants with documented neurologic pharyngeal muscle dysfunction (ALS and muscular dystrophy), and participants with severe OSA. For each participant diagnostic properties of EMG studies will be assessed using a conventional needle and TM-EMG sensor in pharyngeal muscles. The study is designed to test the performance characteristics and capabilities of study design, measures, procedures, recruitment criteria, and operational strategies that are under consideration for use in a larger subsequent study. In other study designs, other NM diseases or study sizes may be selected to provide adequate power to achieve statistical significance of one or more study endpoints.

One objective is to assess or validate the TM-EMG sensor as a non-invasive technique for the assessment of neuromuscular function in the upper airway. Another objective is to elicit, using the TM-EMG sensor, neuromuscular findings that correlate to OSA in affected participants. Although not to any hypothesis, it is hypothesized that TM-EMG examination of muscles located within internal body cavities and organ systems, such as the oropharynx using the TM-EMG sensor, will have similar diagnostic findings to the conventional NEMG examinations and that neurogenic changes will be observed in OSA, or at least achieve certain signal criteria closer to NEMG tracings than SEMG tracings.

The primary endpoint may be proof of diagnostic consistency using both the TM-EMG sensor and NEMG in neuromuscular disorders affecting oropharyngeal muscles. Other endpoints may include Proof that participants with known OSA exhibit EMG findings different from healthy participants and that these findings can be detected using both the NEMG and the TM-EMG sensor.

The participants may be male or female, aged 18 to 70 years old. The participants must be able pause use of anticoagulation, NSAID and multi-vitamins for appropriate period prior to study test, with the timeline and safety to be determined by principal investigator. Exclusion criteria will include:
Allergy to topical anesthetic agent.
Heavy alcohol use—defined as 4 or more alcoholic drinks on the same occasion on 5 or more days in the past month.
Any type of smoking with 10 days prior testing.
The presence of any underlying medical, surgical or psychiatric disorder that would preclude participation in the study as determined study principle investigators.
Prior radiation to the oral cavity.
Craniofacial anatomical disorders.

The study participants will include 8 healthy volunteers and 15 total participants with disorders affecting the function of oropharyngeal muscles. Ten participants will be selected with ALS with the presence of bulbar symptoms, or muscular dystrophy with the presence of bulbar symptoms.

Five participants will be selected with moderate to severe OSA diagnosed with in-laboratory PSG, with the following criteria:
AHI>25 made up of primarily of obstructive apneas and hypopneas
Nadir Sa02<85%
Untreated

The healthy participants will be selected for the following criteria:
Normal craniofacial anatomy
AHI≤2
Epworth score≤10
Stop-Bang score≤2
BMI<30

Each healthy study participants selected for the study will be required to complete an overnight stay for an in-laboratory PSG.

Initial screening of each participant will include demographic information, a comprehensive history including history of present illness (if any), medications, past medical history, allergies, family history, social history—including smoking and alcohol consumption, STOP-Bang, and Epworth sleepiness scale. A comprehensive physical examination will be performed. The data will be documented using Cerner Ambulatory Electronic Health Record. Once a candidate is selected for the trial, they will be presented with the risks and benefits of this study. This will be discussed with each participant prior to written consent being obtained. Risks include induction of a gag reflex and/or feeling of nausea, minor irritation or bleeding at the testing site, mild allergic reactions to the topical anesthetic or to the probe tip itself, infection at the needle insertion site. All reactions, if any, are expected to be minor. Each healthy study participants selected for the study will also be required to complete an overnight stay for an in-laboratory PSG.

Standard ENT examining room equipment and supplies will be available as part of the study. Each participant will be positioned in a powered reclining and elevating otolaryngology examination chair. The subinvestigator will be a first testing physician for each participant. The testing physician will stand to the left of the sitting participant, and the participant will be positioned at the appropriate height, head supported in a headrest, to allow the physician to adequately visualize the soft palate anatomy. The physician will use a headlight to illuminate the oral airway. Photographs of the participant and the participant's soft palate/tongue/dentition and oral airway will be taken, and anatomic findings documented. Participant's blood pressure, heart rate, and respiratory rate will be recorded prior to the start of testing.

The skin of the cheek on the zygomatic arch will be prepared and cleaned with an alcohol swab to optimize skin contact with the ground electrode. The TM-EMG probe device is then attached to the EMG system.

Each participant will open his or her mouth adequately to allow for visualization of the soft palate musculature by the physician. For participants in which the palatopharyngeus/palatoglossus or musculus uvulae are poorly seen, gentle tongue depression will be used to better visualize the anatomy.

Topical 20% Benzocaine will be applied to the mucosal surfaces overlying the muscles to be examined using the TM-EMG probe prior to the start of testing. With the participant's mouth open and in neutral position, the TM-EMG probe is positioned on the mucosal surface correlating to the expected midpoint of the palatoglossus by the subinvestigator. The TM-EMG probe will be used to passively detect and record muscle electrical activity, no energy is applied to the participant through the probe. Free run EMG recording of the palatoglossus will be obtained. Once an optimal or adequate EMG tracing is obtained the probe will be released off the mucosal surface. The same procedure is repeated on the contralateral palatoglossus.

The same protocol will be used to test the genioglossus. The TM-EMG probe is placed on the mucosa of the genioglossus until an optimal EMG tracing is obtained. The same procedure is repeated on the contralateral genioglossus.

The principal investigator will then assess the same muscles. The palatoglossus will be examined in the same location using the same TM-EMG probe. Once an optimal or adequate EMG recording is obtained, the TM-EMG probe will be released off the mucosal surface and an FDA-cleared or approved very fine concentric needle electrode (e.g., Ambu Neuroline 25 mm×30G) will be placed into the palatoglossus using the procedure as described below. Needle insertion will be done in a straight line through the mucosal membrane into the midpoint of the muscle at the desired depth. 1-3 needle insertions will be used to obtain a diagnostic-quality EMG tracing from each muscle. The needle is placed in the same location tested by the TM-EMG probe, Once both tracings are recorded, the contralateral palatoglossus will be tested via both TM-EMG and NEMG. The principal investigator will then obtain bilateral genioglossus EMG using the same protocol that was used for the palatoglossus. Use of the needle electrode in this procedure is consistent with its intended use.

The principal investigator will also obtain EMG data from both left and right palatopharyngeus using the TM-EMG probe and associated protocol. However, due to anticipated difficulty with gag reflex associated with the palatopharyngeus, NEMG will not be used at this site, and testing of this muscle will only be performed by the principal investigator. In total, 6 muscles locations will be tested via TM-EMG, and 4 muscle locations by NEMG.

The free-run EMG tracing for both TM-EMG and NEMG will be displayed on an oscilloscope and heard through an audio-amplifier. All EMG recordings will be made using standard gain and sweep speed settings. Waveforms for both TM-EMG and NEMG tests will be recorded on a Cadwell Sierra Summit EMG system, or other commercially available EMG system.

The recording time per muscle will be approximately 10 seconds. If participant exhibits intolerance during an individual muscle test, that particular muscle test will be discontinued.

Each participant will be examined at the conclusion of the EMG procedures for excessive bleeding or swelling. The participant will have blood pressure, heart rate, and respiratory rate checked for stability immediately upon completion of testing and prior to discharge, Each participant will be given written instructions on how to contact the research staff should any adverse events arise, such as a sore throat, fever, testing site irritation or any other study related concerns. The research staff will contact the participant 1 day and 7 days after completion of the EMG test to document any adverse events the participant may have had following the testing. Responses will be documented in the EMR and serious adverse events will be reported to the study sponsor and IRB within 24 hours. Adverse events which occur more than 5 days after the EMG test will not be considered related to the study intervention, unless the investigator suspects a potential link.

Participants will be withdrawn from the study if they withdraw consent or if the study physician determines that there is a safety risk associated with their participation. If a participant exhibits intolerance during an individual muscle test, that particular muscle test will be discontinued.

All TM-EMG and NEMG data will be de-identified and randomized before subsequent analysis is performed offline. Analysis will include the presence of spontaneous activity (fibrillation potentials and positive sharp waves), MUP morphology (number phases, duration and amplitude) and recruitment pattern. MUP morphology will be analyzed manually. Each recording will be labeled as a normal or abnormal study. Abnormal studies will be classified into active or chronic neurogenic changes or myopathic changes. Results will be compared between the healthy cohort and the neurologic disorders cohort; and between the healthy cohort and the OSA cohort. All study records will be de-identified to maximum capability, and all study charts will be kept at a secured location. The study charts will not contain any protected health information (PHI), and each participant will be identified only according to initials and unique study identifier. Following completion of the trial, all data will be maintained by the tertiary referral clinic. Electronic data will be stored on password-protected computers using the clinic's secure servers. Access to trial data will be available to study investigators, research assistants, and statisticians; however, this data will be de-identified to the maximum of the investigators' capabilities. Finally, each investigator will declare any financial or other conflicts of interests prior to study onset, and at completion. All conflicts of interest will be disclosed to the patient as part of the informed consent.

As a pilot study, a formal sample size calculation was not performed. The results from the pilot study will guide the design of a larger, multi-site study adequately powered to show the diagnostic usefulness of TM-EMG. All data will be examined to determine if there are any missing or non-plausible values, and these will be removed. Summary statistics will be calculated, including means or medians, minimums and maximums, standard deviations, and interquartile ranges. The distribution of EMG findings in (SSA will be presented as descriptive statistics.

Although the foregoing implementations has, for the purposes of clarity and understanding, been described in some detail by of illustration and example, it will be apparent that certain changes and modifications may be practiced, and are intended to fall within the scope of the appended claims. Additionally, it should be understood that the components and characteristics of the devices described herein may be used in any combination, and the methods described herein may comprise all or a portion of the elements described herein. The description of certain elements or characteristics with respect to a specific figure are not intended to be limiting or nor should they be interpreted to suggest that the element cannot be used in combination with any of the other described elements.

We claim:

1. A sensor kit, comprising:
a sensor assembly comprising:
a first electrode;
a second electrode; and
a sensor housing coupling the first and second electrodes, wherein the first and second electrodes project from a surface of the sensor housing for a projection length and are spaced apart by a spacing distance, and a first ratio of the spacing distance to the projection length is between about 0.075:1 and about 1.5:1;
a three-conductor cable, comprising:
a first conductor electrically coupled distally to the first electrode and proximally to a first touch proof, single pole connector;
a second conductor electrically coupled distally to the second electrode and proximally to a second touch proof, single pole connector;
a third conductor with a distal end terminating internally within the cable and a proximal end electrically to a third connector; and
an ground adhesive patch electrode, comprising:
a removable release layer attached to an adhesive;
a fourth proximal end electrically coupled to a fourth touch proof, single pole connector; and
a fifth proximal end electrically coupled to a fifth connector that is configured to attach to the third connector of the sensor assembly.

2. The kit of claim 1, wherein a second ratio of a diameter of the first and second electrodes to the spacing distance is between about 0.2:1 and about 5:1.

3. The kit of claim 1, wherein a third ratio of a diameter of the first and second electrodes to the projection length is between about 0.075:1 and about 1.5:1.

4. The kit of claim 1, wherein the first and second electrodes each comprise a rounded distal end.

5. The kit of claim 1, wherein the first and second electrodes are in parallel.

6. The kit of claim 1, wherein the sensor housing is configured to electrically isolate the first electrode from the second electrode.

7. The kit of claim 1, wherein the first electrode is configured as a reference electrode and the second electrode is configured as an active electrode.

8. The kit of claim 1, wherein the first ratio is between about 0.15:1 and about 0.75:1.

9. The kit of claim 2, wherein the second ratio is between about 0.4:1 and about 2.5:1.

10. The kit of claim 3, wherein the third ratio is between about 0.15:1 and about 0.75:1.

11. The kit of claim 1, wherein the spacing distance is between about 0.2 mm and about 1.0 mm.

12. The kit of claim 1, wherein the projection length is between about 0.5 mm and about 3 mm.

13. A sensor kit, comprising:
a sensor assembly, comprising:
a first electrode;
a second electrode electrically isolated from the first electrode; and
a sensor housing coupling the first and second electrodes, the first and second electrodes project in parallel from a surface of the sensor housing, and a distance between central longitudinal axes of the first and second electrodes is between about 0.30 mm and about 2.0 mm; and
a three-conductor cable, comprising:
a first conductor electrically coupled distally to the first electrode and proximally to a first touch proof, single pole connector;
a second conductor electrically coupled distally to the second electrode and proximally to a second touch proof, single pole connector;
a third conductor with a distal end terminating internally within the cable and a proximal end electrically to a third connector; and
an ground adhesive patch electrode, comprising:
a removable release layer attached to an adhesive;
a fourth proximal end electrically coupled to a fourth touch proof, single pole connector; and
a fifth proximal end electrically coupled to a fifth connector that is configured to attach to the third connector of the sensor assembly.

14. The kit of claim 13, wherein the first and second electrodes project from the surface of the housing for a projection length between about 0.5 mm and about 3 mm.

15. The kit of claim 13, wherein a diameter of the first and second electrodes is between. about 0.1 mm and about 1.0 mm.

16. The kit of claim 13, wherein the distance is between about 0.60 mm and about 1.5 mm.

17. The kit of claim 1 or 13, further comprising a probe comprising one or more of the sensors and a handle portion.

18. The kit of claim 17, wherein the probe comprises a first portion and a second portion detachably attached to the first portion.

19. The kit of claim 18, wherein. the first portion. comprises a paddle shape and a radius of curvature of between about 10 cm and about 20 cm.

20. The kit of claim 17, wherein adjacent sensors are spaced apart from each other between about 0.5 cm and about 5 cm.

21. The kit of claim 17, wherein the probe comprises one or more dental markers.

22. The kit of claim 17, wherein the probe further comprises a rigid catheter.

23. The kit of claim 17, wherein the probe further comprises a flexible catheter.

24. The kit of claim 17, further comprising:
an amplifier coupled to the probe; and
a controller coupled to the probe and the amplifier, the controller comprising processor and a memory, and the controller configured to:
receive signal data corresponding to electrical activity of muscle tissue using the one or more sensors;
amplify the signal data; and
generate electromyography data using the amplified signal data.

25. The kit of claim 24, wherein the amplifier comprises a pre-amplifier.

26. The kit of claim 1 or 13, wherein the sensor comprises one or more ground electrodes.

27. The kit of claims 1 or 13, wherein the sensor comprises one or more lead wires coupled to the electrodes, the lead wires comprising between about 7 strands and about 100 strands.

28. The kit of claims 1 or 13, wherein the assembly is configured to receive signal data corresponding to a motor unit action potential having a rise time of less than about 500 μs while the assembly elastically deforms an intact tissue surface without penetrating or piercing the intact tissue surface.
